(19) United States
(12) Patent Application Publication
(10) Pub. No.: US 2016/0376564 A1
(43) Pub. Date: Dec. 29, 2016

(54) Nucleic Acids and Polypeptides for Utilizing Plant Biomass

(71) Applicant: MetaMixis, Inc., Palo Alto, CA (US)

(72) Inventors: Cameron Strachan, Vancouver (CA); Steven Hallam, Vancouver (CA)

(73) Assignee: MetaMixis, Inc., Palo Alto, CA (US)

(21) Appl. No.: 15/191,482

(22) Filed: Jun. 23, 2016

Related U.S. Application Data
(60) Provisional application No. 62/185,377, filed on Jun. 26, 2015.
(60) Provisional application No. 62/191,966, filed on Jul. 13, 2015.

(51) Int. Cl. C12N 9/02; C12N 1/20; C12N 15/70; C07K 14/195

(52) U.S. Cl. CPC C12N 9/0004; C07K 14/195; C12N 1/20; C12N 15/70

(57) ABSTRACT

The present invention relates to novel nucleic acids, novel groups of polypeptides encoded by the polynucleotides, novel compositions, and methods of using the same with lignin containing substrates.

### FIELD OF THE INVENTION

This invention relates to the field of biomass utilization. In particular, the invention relates to nucleic acids and polypeptides useful for utilizing lignin-containing biomass.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “MM009_ST25.txt”, a creation date of Jun. 17, 2016, and a size of 755 kilobytes. The Sequence Listing filed via EFS-Web is part of the specification and is incorporated in its entirety by reference herein.

### BACKGROUND OF THE INVENTION

Lignin is the second most abundant biopolymer on earth and a promising feedstock for deriving energy and industrial chemical precursors from renewable plant resources. The synthesis of lignin occurs within plant cell walls by free radical reactions that cross-link diverse combinations of monoaromatic compounds into a heterogeneous matrix that is resistant to microbial and chemical assailment. Lignin recalcitrance is further reflected in the deposition of coal throughout the Carboniferous period prior to the emergence of fungal enzymes associated with lignolysis in Permian forest soil ecosystems. Although a few bacterial strains and enzymes capable of lignin transformation have been identified, including Enterobacter lignolyticus SCF 1 and Rhodococcus jostii RHA1, white-rot basidiomycetes are currently the major source of lignin transforming enzymes, including laccases, manganese-dependent peroxidases, and lignin peroxidases. This presents numerous technical challenges associated with the genetic tractability of fungal systems and the expression of fungal-derived enzymes in heterologous hosts such as E. coli. Implementing high-throughput methods to expedite the discovery of bacterial lignin transformation pathway components provides one promising route toward overcoming these challenges. However, to date efforts to develop such functional screens have been unreliable due to the inherent complexity of the lignin polymer.

It has long been appreciated that environmental micro-organisms are an excellent source of solutions to industrial problems. In particular, they may provide a source for enzymes and associated co-factors. However, there is also an awareness that environmental microorganisms can be difficult to culture in the laboratory let alone on an industrial scale. Accordingly, a number of metagenome screening methods have been developed to isolate useful genesfrom metagenomes. For example, metagenomic nucleotide sequencing methods (Okuta et al. Gene (1998) 212:221-228), and enzyme activity based screening (Henne et al. Appl. Environ. Microbiol. (1999) 65:3901-3907). Further enzyme activity based screening methods have been developed, such as Substrate-Induced Gene-Expression (SIGEX) screening (Uchiyama et al. Nature Biotechnology(2005) 23(1):88-93) and more recently Product-Induced Gene-Expression (PIGEX) screening (Uchiyama and Miyazaki Appl. Environ. Microbiol. (2010) 76(21):7029-7035). Furthermore, several screening strategies have been developed to discover genetic elements that are activated in response to a metabolite, including intragenic genomic libraries and promoter traps (Uchiyama and Miyazaki PLOS ONE (2013) 8(9):e75795).

### SUMMARY OF THE INVENTION

The present invention relates to nucleic acids and polypeptides useful in lignin utilization. In some embodiments, the invention relates to the nucleic acids, and polypeptides encoded by Fosmid_182_02_CO3 (KJ802934); Fosmid 182_06_L14 (KJ802935); Fosmid 182_07_CO2 (KJ802936); Fosmid 182_08_C21 (KJ802937); Fosmid_182_09_J11 (KJ802938); Fosmid_182_10_L09 (KJ802939); Fosmid_182_11_B22 (KJ802940); Fosmid_182_13_A07 (KJ802941); Fosmid_182_13_F13 (KJ802942); Fosmid_182_16_E12 (KJ802943); Fosmid_182_16_J11 (KJ802944); Fosmid_182_17_09 (KJ802945); Fosmid_182_19_A11 (KJ802946); Fosmid_182_35_020 (KJ802947); Fosmid_182_42_K21 (KJ802948); Fosmid_183_01_D18 (KJ802949); Fosmid 183_12_O16 (KJ802950); Fosmid_183_21_D14 (KJ802951); Fosmid_183_24_C18 (KJ802952); Fosmid_183_26_G23 (KJ802953); Fosmid_183_29_MO4 (KJ802954); Fosmid_183_38_D19 (KJ802955); Fosmid_183_42_E18 (KJ802956); and Fosmid 183_52_O2 (KJ802957).

In some embodiments, the invention related to nucleic acids of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, or 75. In some embodiments, the invention relates to polypeptides of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, or 76. In some embodiments, the invention relates to electron transfer polypeptides (e.g., oxidoreductase activity) of SEQ ID NOS: 2, 12, 14, 24, 30, 36, 38, 50, 56, 62, 64, 68, 70, and 72, and/or co-factor generation polypeptides (e.g., hydrogen peroxide formation) of SEQ ID NOS: 4, 16, 28, 48, and 60, protein secretion polypeptides (e.g., secretion apparatus or signal peptide) of SEQ ID NOS: 6, 20, 32, 42 and 44, and polypeptides involved in small molecule transport (e.g., multidrug efflux superfamily) of SEQ ID NO: 34. In some embodiments, the nucleic acids and polypeptides of the invention are motility related polypeptides (e.g., methyl accepting chemotaxis proteins (MCP), or signal transduction pathway components (e.g., PAS domain containing sensors). In some embodiments, the nucleic acids and/or polypeptides of the invention include variants or analogs or alleles of any of the above nucleic acids. In some embodiments, the nucleic acids of the invention hybridize under stringent hybridization conditions to one of SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, or 75. In some embodiments, the polypeptides of the invention are encoded by nucleic acids that hybridize under stringent hybridization conditions to one of SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, or 75. In some embodiments, the polypeptides of the invention have 70%, 80%, 90%, or 95% sequence identity with a polypeptide of SEQ ID NO. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, or 76.

In some embodiments, the invention also relates to nucleic acids of SEQ ID NOs: 77-96. In some embodiments, the invention relates to polypeptides found in Table 3 that are encoded within SEQ ID NOs: 77-96. In some embodiments, the nucleic acids and/or polypeptides of the invention include variants or analogs or alleles of any of the above nucleic acids and/or polypeptides. In some embodiments, the nucleic acids of the invention hybridize under stringent hybridization conditions to one of SEQ ID NO. 77-96. In some embodiments, the polypeptides of the invention are encoded by nucleic acids that hybridize under stringent hybridization conditions to one of SEQ ID NO. 77-96. In some embodiments, the polypeptides of the invention have 70%, 80%, 90%, or 95% sequence identity with a polypeptide of Table 3 that are encoded within SEQ ID NOs: 77-96.

In some embodiments, host cells contain the nucleic acids and/or polypeptides of the invention. In some embodiments, the host cells are prokaryotic cells. In some embodiments, the prokaryotic cell is a species from Acidovorax, Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. In some embodiments, the host cell is E. coli. In some embodiments, the host cell is a eukaryotic cell. In some embodiments, the eukaryotic cell is an algae specie and/or a photosynthetic microorganism from Agmenellum, Amphora, Anabaena, Ankistrodesmus, Botryococcus, Boekelovia, Borodinella, Botryococcus, Carteria, Chaetoceros, Chlamydomonas, Chlorella, Chlorococcum, Chlorogonium, Chrysosphaera, Cricosphaera, Cryptomonas, Cyclotella, Dunaliella, Ellipsoidon, Eremosphaera, Euglena, Fragilaria, Gleocapsa, Gloeothamnion, Hymenomonas, Isochrysis, Lepocinclis, Monoraphidium, Nannochloris, Nannochloropsis, Navicula, Nephrochloris, Nitschia, Nitzschia, Ochromonas, Oocystis, Oscillatoria, Nitzschia, Pascheria, Phagus, Phormidium, Platymonas, Pleurochrysis Prototheca, Pyrobotrys Scenedesmus, Spirogyra, Tetraedron, Tetraselmis, or Volvox. In some embodiments, the host cell is Botryococcus braunii, Prototheca krugani, Prototheca moriformis, Prototheca portoricensis, Prototheca stagnora, Prototheca wickerhamii, or Prototheca zopfii. In some embodiments, the eukaryotic cell is a fungi specie from Aspergillus, Candida, Chlamydomonas, Chrysosporium, Cryotococcus, Debaromyces, Fusarium, Hansenula, Kluyveromyces, Neotyphodium, Neurospora, Penicillium, Pichia, Saccharomyces, Schizosaccharomyce, Trichoderma, Xanthophyllomyces, Yarrowia, and Zygosaccharomyces. In some embodiments, the fungi is Pichia pastoris, Saccharomyces cerevisiae, Schizosaccharomyces pompe, Kluyveromyces lactic, Hansenula polymorpha, or a filamentous fungi, e.g. Trichoderma, Aspergillus sp., including Aspergillus niger, Aspergillus phoenicis, Aspergillus carbonarius.

In some embodiments, the invention relates to extracts made from host cells containing the nucleic acids and/or polypeptides of the invention. In some embodiments, the extracts are made from E. coli that contain one or more of the fosmids of the invention. In some embodiments, the extracts are made from E. coli containing one or more of the fosmids: Fosmid_182_35_O20 (Annotation No. KJ802947), Fosmid 182_16_J11 (Ann. No. KJ802944), Fosmid_182_11_B22 (Ann. No. KJ802940), Fosmid 182_09_J11 (Ann. No. KJ802938), Fosmid_182_42_K21 (Ann. No. KJ802948), Fosmid 182_02_CO3 (Ann. No. KJ802934), or Fosmid_182_16_E12 (Ann. No. KJ802943). In some embodiments, the extracts are made from E. coli that contains one or more of SEQ ID NOs.: 77-96. In some embodiments, the extracts contain one or more of the polypeptides from Table 3 that are encoded within SEQ ID NOs: 77-96. In some embodiments, the extracts are made from host cells containing one or more of these nucleic acids and/or polypeptides, and additionally containing other nucleic acids of the invention. In some embodiments, the invention relates to a mixture of extracts comprising extracts made from host cells with one or more nucleic acids and/or polypeptides of the invention, and extracts made from cells without a nucleic acid or polypeptide of the invention.

In some embodiments, the invention relates to methods for utilizing lignin containing biomass or substrates using the nucleic acids and/or polypeptides of the invention. In some embodiments, extracts made from host cells containing the nucleic acids of the invention are used for lignin utilization. In some embodiments, lignin containing biomass or substrates are combined with a mixture of extracts comprising extracts made from host cells with one or more nucleic acids and/or polypeptides of the invention, and extracts made from cells without a nucleic acid or polypeptide of the invention. In some embodiments, host cells containing the nucleic acids and/or polypeptides of the invention are used with lignin containing biomass or substrates.

In some embodiments, the invention relates to combinatorial use of nucleic acids and/or polypeptides for a non-heme bacterial or archaeal oxidoreductase that binds Fe/Cu/Zn/Mn and utilizes lignin or lignin transformation products as a substrate and one or more bacterial proteins from functional classes (a) to (e): (a) co-substrate generation; (b) protein secretion; (c) small molecule or breakdown product transportation or bacterial efflux pumps and related transmembrane proteins; (d) motility and protein secretion machinery; and (e) signal transduction or transcriptional regulation; for transforming a heterogeneous aromatic polymer.

In some embodiments, the invention relates to a combinatorial use of nucleic acids and/or polypeptides for a non-heme bacterial or archaeal oxidoreductase that binds Fe/Cu/Zn/Mn and utilizes heterogeneous aromatic polymers or their transformation products as a substrate and one or more bacterial proteins from functional classes (a) to (e): (a) co-substrate generation; (b) protein secretion; (c) small molecule or breakdown product transportation or bacterial efflux pumps and related transmembrane proteins; (d) motility and protein secretion machinery; and (e) signal transduction or transcriptional regulation; for transforming a heterogeneous aromatic polymer.

In some embodiments, the invention relates to a method for transforming a heterogeneous aromatic polymer, the method including: (a) the addition of a non-heme bacterial or archaeal oxidoreductase that binds Fe/Cu/Zn/Mn and utilizes lignin or lignin transformation products as a substrate to a heterogeneous aromatic polymer source; and (b) the addition of one or more bacterial or archaeal proteins from the functional classes (i) to (v): (i) co-substrate generation; (ii) protein secretion; (iii) small molecule or breakdown product transportation or bacterial efflux pumps; (iv) motility and protein secretion machinery; and (v) signal transduction or transcriptional regulation.

In some embodiments, the invention relates to a method for transforming a heterogeneous aromatic polymer, the method including: (a) the addition of a non-heme bacterial or archaeal oxidoreductase that binds Fe/Cu/Zn/Mn and utilizes heterogeneous aromatic polymers or their transformation products as a substrate to a heterogeneous aromatic polymer source; and (b) the addition of one or more bacterial or archaeal proteins from the functional classes (i) to (v): (i) co-substrate generation; (ii) protein secretion; (iii) small molecule or breakdown product transportation or bacterial efflux pumps; (iv) motility and protein secretion machinery; and (v) signal transduction or transcriptional regulation.

In some embodiments, the invention relates to a method for heterogeneous aromatic polymer transformation, the method including: (a) obtaining a heterogeneous aromatic polymer source material; and (b) adding an archaebacterial or bacterialorganism to the heterogeneous aromatic polymer source material from (a), wherein the archaebacteria or bacteria comprises a combination of protein-coding genes selected from a non-heme bacterial or archaeal oxidoreductase that binds Fe/Cu/Zn/Mn and utilizes lignin or lignin transformation products as a substrate to a heterogeneous aromatic polymer source; and one or more bacterial or archaeal of protein-coding genes from the functional classes (i) to (v): (i) co-substrate generation; (ii) protein secretion; (iii) small molecule or breakdown product transportation or bacterial efflux pumps; (iv) motility and protein secretion machinery; and (v) signal transduction or transcriptional regulation, in an amount sufficient to and for a sufficient time period to cause transformation of the heterogeneous aromatic polymer to a desired product.

In some embodiments, the invention relates to In accordance with another aspect of the invention, there is provided a method for heterogeneous aromatic polymer transformation, the method including: (a) obtaining a heterogeneous aromatic polymer source material; and (b) adding an archaebacterial or bacterial organism to the heterogeneous aromatic polymer source material from (a), wherein the archaebacteria or bacteria comprises a combination of protein-coding genes selected from a non-heme bacterial or archaeal oxidoreductase that binds Fe/Cu/Zn/Mn and utilizes heterogeneous aromatic polymers or their transformation products as a substrate to a heterogeneous aromatic polymer source; and one or more bacterial or archaeal of protein-coding genes from the functional classes (i) to (v): (i) co-substrate generation; (ii) protein secretion; (iii) small molecule or breakdown product transportation or bacterial efflux pumps; (iv) motility and protein secretion machinery; and (v) signal transduction or transcriptional regulation, in an amount sufficient to and for a sufficient time period to cause transformation of the heterogeneous aromatic polymer to a desired product.

### BRIEF DESCRIPTION OF THE DRAWINGS

FIG. 1 illustrates a profile of relative amounts of monoaromatic compounds detected by GC-MS, for cultures of E. coli harboring different fosmid clones after incubation with HKL-F1 in minimal media.

FIG. 2 illustrates a profile of relative amounts of monoaromatic compounds detected by GC-MS, for cultures of E. coli harboring different fosmid clones after incubation with HP-L™ in minimal media.

FIG. 3 illustrates a comparative analysis of gene types in active fosmids, wherein the bar graphs show the relative number of annotated genes falling within the six functional classes implicated in lignin transformation phenotypes.

### DETAILED DESCRIPTION OF THE INVENTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### DEFINITIONS

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, “biomass” refers to material produced by growth and/or propagation of cells. Biomass may contain cells and/or intracellular contents as well as extracellular material.

As used herein, “codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome.

As used herein, “consensus sequence” and “canonical sequence” refer to an archetypical amino acid sequence against which all variants of a particular protein or sequence of interest are compared. The terms also refer to a sequence that sets forth the nucleotides that are most often present in a DNA sequence of interest among members of related gene sequences. For each position of a gene, the consensus sequence gives the amino acid that is most abundant in that position in a multiple sequence alignment (MSA).

As used herein, “control sequence” refers to components, which are used for the expression of a polynucleotide and/or polypeptide of the present invention. Each control sequence may be native or foreign to the nucleic acid sequence encoding the polypeptide. Such control sequences may include, but are not limited to, some or all of the following: a promoter, an enhancer, an operator, an attenuator, a shine-delgarno sequence, a leader, a polyadenylation sequence, a propeptide sequence, a signal peptide sequence, and a transcription terminator. At a minimum, the control sequences include a promoter and transcriptional signals, and where appropriate, translational start and stop signals.

As used herein, an “effective amount” refers to an amount of a compound, formulation, material, or composition, as described herein effective to achieve a particular biological result.

As used herein, the terms “expression vector” or “expression construct” or “plasmid” or “recombinant DNA construct” refer to a nucleic acid construct, that has been generated recombinantly or synthetically via human intervention, including by recombinant means or direct chemical synthesis, with a series of specified nucleic acid elements that permit transcription and/or translation of a particular nucleic acid in a host cell. The expression vector can be part of a plasmid, virus, or nucleic acid fragment. Typically, the expression vector includes a nucleic acid to be transcribed operably linked to a promoter. The expression vector can exist in a host cell in either an episomal or integrated vector.

As used herein, “exogenous gene” refers to a nucleic acid that codes for the expression of an RNA and/or protein that has been introduced (“transformed”) into a cell. A transformed cell may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome or as an episomal molecule.

As used herein, “extract” refers to a solution containing the contents or sub-contents of lysed cells.

As used herein, “heterologous” polynucleotide or polypeptide refers to any polynucleotide that is introduced into a host cell by laboratory techniques, or a polynucleotide that is foreign to a host cell. As such, the term includes polynucleotides that are removed from a host cell, subjected to laboratory manipulation, and then reintroduced into a host cell. In some embodiments, the introduced polynucleotide expresses the heterologous polypeptide. Heterologous polypeptides are those polypeptides that are foreign to the host cell being utilized.

As used herein, “isolated polypeptide” refers to a polypeptide which is substantially separated from other components that naturally accompany it, e.g., protein, lipids, and polynucleotides. The term embraces polypeptides which have been removed or purified from their naturally-occurring environment or expression system (e.g., host cell or in vitro synthesis). The engineered polypeptides of the invention may be present within a cell, present in the cellular medium, or prepared in various forms, such as lysates or isolated preparations.

As used herein, “lysis” refers to the breakage of the plasma membrane and optionally the cell wall of a biological organism sufficient to release at least some intracellular content, often by mechanical, viral or osmotic mechanisms that compromise its integrity.

As used herein, “lysing” refers to disrupting the cellular membrane and optionally the cell wall of a biological organism or cell sufficient to release at least some intracellular content.

As used herein, “naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “operably linked” and “operable linkage” refer to a configuration in which a control sequence or other nucleic acid is appropriately placed (i.e., in a functional relationship) at a position relative to a polynucleotide of interest such that the control sequence or other nucleic acid can interact with the polynucleotide of interest. In the case of a control sequence, operable linkage means the control sequence directs or regulates the expression of the polynucleotide and/or polypeptide of interest. In the case of polypeptides, operably linked refers to a configuration in which a polypeptide is appropriately placed at a position relative to a polypeptide of interest such that the polypeptide can interact as desired with the polypeptide of interest.

As used herein, “percentage of sequence identity” and “percentage homology” are used interchangeably herein to refer to comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison Wis.), using default parameters provided.

As used herein, “recombinant” or “engineered” or “non-naturally occurring” refers to a cell, nucleic acid, protein or vector that has been modified due to the introduction of an exogenous nucleic acid or the alteration of a native nucleic acid. Thus, e.g., recombinant cells express genes that are not found within the native (non-recombinant) form of the cell or express native genes differently than those genes are expressed by a non-recombinant cell. A “recombinant nucleic acid” is a nucleic acid originally formed in vitro, in general, by the manipulation of nucleic acid, e.g., using polymerases and endonucleases, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid.

As used herein, “reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes to the primary sequence.

As used herein, “saccharification” refers to a process of converting biomass, usually cellulosic or lignocellulosic biomass, into monomeric sugars, such as glucose and xylose. “Saccharified” or “depolymerized” cellulosic material or biomass refers to cellulosic material or biomass that has been converted into monomeric sugars through saccharification.

As used herein, “stringent hybridization conditions” refers to hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/O. 1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As used herein, “substantial identity” refers to a polynucleotide or polypeptide sequence that has at least 80 percent sequence identity, at least 85 percent identity and 89 to 95 percent sequence identity. Substantial identity also encompasses at least 99 percent sequence identity as compared to a reference sequence over a comparison window of at least 20 residue positions or a window of at least 30-50 residues, wherein the percentage of sequence identity is calculated by comparing the reference sequence to a sequence that includes deletions or additions or substitutions over the window of comparison. In specific embodiments applied to polypeptides, the term “substantial identity” means that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity).

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

### Lignin Utilizing Polynucleotides, Polypeptides, and Extracts

In one aspect, the invention provides polypeptides having activities that improve lignin utilization, including, polypeptides with electron transfer activity (e.g., oxidoreductase activity), polypeptides involved with co-factor generation (e.g., hydrogen peroxide formation), polypeptides involved with protein secretion (secretion apparatus or signal peptide), polypeptides involved with small molecule transport (e.g., multidrug efflux superfamily), polypeptides involved with motility (e.g., methyl accepting chemotaxis proteins (MCP)), and polypeptides involved with signal transduction pathway components (e.g., PAS domain containing sensors).

In some embodiments, one or more nucleic acids having the sequences of SEQ ID NOs: 77-96 are used in the invention to utilize lignin containing biomass or substrates. Tables 1 and 3 lists some of the polynucleotides of the invention, and describes the Fosmid ID, Island number within the Fosmid (some fosmids have two genetics islands), GenBank Accession number, start and stop sequences, and SEQ ID NO. Table 1 and 3 lists the Fosmid ID, Island number, GenBank Accession number, start and stop sequences, and description for each polypeptide encoded by a SEQ ID NO and Island of the invention.

The present invention also relates to recombinant and/or isolated and/or purified polypeptide sequences that are selected from a polypeptide sequence or a fragment of a polypeptide sequence of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, or 76. Tables 1 lists the Gene ID, Accession No., polypeptide class, secretion signal class, polynucleotide SEQ ID NO., and polypeptide SEQ ID NO. for exemplary nucleic acids of the invention. All of the sequences reported in Tables 1 and 3, and all the sequences reported at the above GenBank Accession numbers are hereby incorporated by reference in their entirety for all purposes. The functional groups are: electron transfer (e.g., oxidoreductase activity) for example SEQ ID NOS: 2, 12, 14, 24, 30, 36, 38, 50, 56, 62, 64, 68, 70, and 72), co-factor generation (e.g., hydrogen peroxide formation) for example SEQ ID NOS: 4, 16, 28, 48, and 60), protein secretion (e.g., secretion apparatus or signal peptide) for example SEQ ID NOS: 6, 20, 32, 42, and 44), and small molecule transport (e.g., multidrug efflux superfamily) for example SEQ ID NOS: 34. Other functional groups into which nucleic acids of the Tables fall are: motility (e.g., methyl accepting chemotaxis proteins (MCP), and signal transduction pathway components (e.g., PAS domain containing sensors). In some embodiments, one or more of the lignin utilization polypeptides are used. In some embodiments, the one or more lignin utilization polypeptides are used in a host cell.

The present invention also relates to recombinant and/or isolated and/or purified polypeptide sequences that are selected from a polypeptide sequence or a fragment of a polypeptide sequence of the polypeptide and nucleotide sequences found in the sequences deposited at GenBank under Accession Nos. KJ802937, KJ802939, KJ802940, KJ802942, KJ802943, KJ802944, KJ802947, KJ802948, KJ802949, KJ802951, KJ802953, and KJ802957, and other deposited sequences are found at Accessions Nos. KJ802934, KJ802935, KJ802936, KJ802937, KJ802938, KJ802939, KJ802940, KJ802941, KJ802942, KJ802943, KJ802944, KJ802945, KJ802946, KJ802947, KJ802948, KJ802949, KJ802950, KJ802951, KJ802952, KJ802953, KJ802954, KJ802955, KJ802956, and KJ802957, and exemplary descriptions of these sequences are found in Tables 1 and 3. All of the sequences reported in Table 1 and 3, and all the sequences reported at the above GenBank Accession Nos. are incorporated by reference in their entirety for all purposes. The functional groups are: electron transfer (e.g., oxidoreductase activity), co-factor generation (e.g., hydrogen peroxide formation), protein secretion (e.g., secretion apparatus or signal peptide), and small molecule transport (e.g., multidrug efflux superfamily), motility (e.g., methyl accepting chemotaxis proteins (MCP), and signal transduction pathway components (e.g., PAS domain containing sensors). In some embodiments, one or more of the lignin utilization polypeptides are used. In some embodiments, the one or more lignin utilization polypeptides are used in a host cell.

The following fosmids sequences were deposited on 8 May 2014, and published on Jul. 14, 2014, in association with the corresponding accession numbers as follows: Fosmid_182_02 CO3 (KJ802934); Fosmid_182_06_L14 (KJ802935); Fosmid 182_07_CO2 (KJ802936); Fosmid_182_08_C21 (KJ802937); Fosmid_182_09_J11 (KJ802938); Fosmid_182_10 L09 (KJ802939); Fosmid_182_11_B22 (KJ802940); Fosmid_182_13_A07 (KJ802941); Fosmid_182_13_F13 (KJ802942); Fosmid 182_16_E12 (KJ802943); Fosmid_182_16_J11 (KJ802944); Fosmid_182_17_09 (KJ802945); Fosmid_182_19_A11 (KJ802946); Fosmid_182_35_020 (KJ802947); Fosmid_182_42_K21 (KJ802948); Fosmid_183_01_D18 (KJ802949); Fosmid 183_12_O16 (KJ802950); Fosmid_183_21_D14 (KJ802951); Fosmid_183_24_C18 (KJ802952); Fosmid_183_26_G23 (KJ802953); Fosmid_183_29_MO4 (KJ802954); Fosmid_183_38_D19 (KJ802955); Fosmid_183_42_E18 (KJ802956); and Fosmid 183_52_O2 (KJ802957). These Fosmid sequences are hereby incorporated by reference in their entirety for all purposes.

The present invention also relates to extracts made from host cells comprising and expressing the polypeptides of the invention. In some embodiments, the polypeptides from the invention are expressed in a host organism that could be a representative of taxonomic groups such as Acidovorax, Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. In some embodiment, these host organisms are grown and one or more of the polypeptides related to this invention are extracted. For example, a polypeptide from fosmid ID 182_16_J11 with gene ID 182_16_J11_2 that is related to aromatic hydrocarbon degradation is overexpressed in Eschrichia coli. In another example, a copper binding protein related to a polypeptide from fosmid ID 182_08_C21 was expressed in Eschrichia coli. In other examples, a genomic island spanning from approximately 169-6997 on fosmid 182_35_O20 could be expressed in Eschrichia coli. The polypeptides from E. coli cells harboring the genomic island can be extracted and used to modify a heterogeneous aromatic polymer to improve cellulose conversion. In some embodiments, the genomic islands or resulting polypeptides are modified to change the level of expression in the host organism. These modifications include, but are not limited to, mutations, nucleotide insertions, gene synthesis and sub cloning. These host organisms harboring genomic islands are grown and one or more of the polypeptides related to this invention are extracted.

The extracts may include native polypeptides or other molecules from the host organism. In some embodiments, mixtures of extracts are used and the mixture comprises or one or more extracts made from host cells expressing nucleic acids of the invention and optionally, extracts made from cells that do not contain a nucleic acid of the invention. In some embodiments, the polypeptides can be released from the host organism by physical or chemical methods. This may include, but is not limited to, the use of organic solvents, surfactants or enzymes such as lysozyme. Enrichment or concentration steps can be conducted using, but is not limited to, affinity chromatography, porous membranes or centrifugation, or other standard and well-known procedures in the art for enriching, separating and/or concentrating desired polypeptides or factors. See, for example, Sambrook et al., Molecular Cloning: A Laboratory Manual, Cold Spring Harbor Laboratory Press, Cold Spring Harbor, N.Y. (1989); Current Protocols in Molecular Biology, Ausubel et al., eds, Green Publishers Inc. and Wiley and Sons, N.Y (1994); Scopes, R. K., Protein Purification: Principles and Practice, Springer Advanced Texts in Chemistry (3^{rd} Ed., 1993), each of which is incorporated by reference in its entirety for all purposes. In one embodiment, the polypeptides from E. coli were released by chemical means that include detergents such as SDS. The soluble polypeptides can subsequently be concentrated and exchanged into alternate buffers. In some embodiments, one or more purified proteins, partially purified proteins, other extracts, and/or small molecule mediators are added to the extracts of the invention. This can include, but is not limited by, bacterial laccases and small molecule mediators such as ABTS. In some embodiments, the extracts are used to modify a heterogeneous aromatic polymer and improve cellulose conversion.

In some embodiments, the step of lysing a host cell to make an extract comprises lysing the microorganism by using an enzyme. In some embodiments, enzymes for lysing a microorganism are proteases and polysaccharide-degrading enzymes such as hemicellulase (e.g., hemicellulase from Aspergillus niger; Sigma Aldrich, St. Louis, Mo.; #H2125), pectinase (e.g., pectinase from Rhizopus sp.; Sigma Aldrich, St. Louis, Mo.; #P2401), Mannaway 4.0 L (Novozymes), cellulase (e.g., cellulose from Trichoderma viride; Sigma Aldrich, St. Louis, Mo.; #C9422), and driselase (e.g., driselase from Basidiomycetes sp.; Sigma Aldrich, St. Louis, Mo.; #D9515. In some embodiments, the enzymes include for example, a cellulase such as a polysaccharide-degrading enzyme, optionally from Chlorella or a Chlorella virus, or a proteases, such as Streptomyces griseus protease, chymotrypsin, proteinase K, proteases listed in Degradation of Polylactide by Commercial Proteases, Oda Y et al., Journal of Polymers and the Environment, Volume 8, Number 1, January 2000, pp. 29-32(4), Alcalase 2.4 FG (Novozymes), and Flavourzyme 100 L (Novozymes), Oda et al is hereby incorporated by reference in its entirety for all purposes. Any combination of a protease and a polysaccharide-degrading enzyme can also be used, including any combination of the preceding proteases and polysaccharide-degrading enzymes.

In some embodiments, lysis is performed using an expeller press. In this process, host cells are forced through a screw-type device at high pressure, lysing the cells and causing the intracellular contents of the host cells to be released and separated from the membranes and fiber (and other components) in the cell.

In some embodiments, the step of lysing the host cell is performed by using ultrasound, i.e., sonication. Thus, host cells can also be lysed with high frequency sound. The sound can be produced electronically and transported through a metallic tip to an appropriately concentrated cellular suspension. This sonication (or ultrasonication) disrupts cellular integrity based on the creation of cavities in the cell suspension.

In some embodiments, the step of lysing the host cells is performed by mechanical lysis. Host cells can be lysed mechanically and optionally homogenized to facilitate extract collection. For example, a pressure disrupter can be used to pump a host cell containing slurry through a restricted orifice valve. High pressure (up to 1500 bar) is applied, followed by an instant expansion through an exiting nozzle. Host cell disruption is accomplished by three different mechanisms: impingement on the valve, high liquid shear in the orifice, and sudden pressure drop upon discharge, causing an explosion of the host cell. The method releases intracellular molecules. Alternatively, a ball mill can be used. In a ball mill, host cells are agitated in suspension with small abrasive particles, such as beads. Host cells break because of shear forces, grinding between beads, and collisions with beads. The beads disrupt the host cells to release cellular contents. Host cells can also be disrupted by shear forces, such as with the use of blending (such as with a high speed or Waring blender as examples), the French press, or even centrifugation in case of weak cell walls, to disrupt host cells. In some embodiments, the step of lysing a host cell is performed by applying an osmotic shock.

In some embodiments, the step of lysing a microorganism comprises infecting the host cell with a lytic virus. A wide variety of viruses are known to lyse host cells of the invention and are suitable for use in the present invention. The selection and use of a particular lytic virus for a particular host cell is within the level of skill in the art. For example, paramecium bursaria chlorella virus (PBCV-1) is the prototype of a group (family Phycodnaviridae, genus Chlorovirus) of large, icosahedral, plaque-forming, double-stranded DNA viruses that replicate in, and lyse, certain unicellular, eukaryotic chlorella-like green algae. Accordingly, any susceptible microalgae can be lysed by infecting the culture with a suitable chlorella virus. Methods of infecting species of Chlorella with a chlorella virus are known. See for example Adv. Virus Res. 2006; 66:293-336; Virology, 1999 Apr. 25; 257(1):15-23; Virology, 2004 Jan. 5; 318(1):214-23; Nucleic Acids Symp. Ser. 2000; (44):161-2; J. Virol. 2006 March; 80(5):2437-44; and Annu. Rev. Microbiol. 1999; 53:447-94, all of which are hereby incorporated by reference in their entirety for all purposes.

In some embodiments, the step of lysing a host cell comprises autolysis. In this embodiment, a host cell is genetically engineered to produce a lytic protein that will lyse the host cell at a desired time. This lytic gene can be expressed using an inducible promoter so that the cells can first be grown to a desirable density in a incubator or other container, followed by induction of the promoter to express the lytic gene to lyse the cells. In one embodiment, the lytic gene encodes a polysaccharide-degrading enzyme. In certain other embodiments, the lytic gene is a gene from a lytic virus. Thus, for example, a lytic gene from a Chlorella virus can be expressed in an algal cell; see Virology 260, 308-315 (1999); FEMS Microbiology Letters 180 (1999) 45-53; Virology 263, 376-387 (1999); and Virology 230, 361-368 (1997), all of which are hereby incorporated by reference in their entirety for all purposes. Expression of lytic genes is preferably done using an inducible promoter, such as a promoter active in the host cell that is induced by a stimulus such as the presence of a small molecule, light, heat, and other stimuli.

In some embodiments, the polynucleotides and/or polypeptides are modified to change the level of expression in the host organism. These modifications include, but are not limited to, mutations, nucleotide insertions, gene synthesis and sub cloning.

The polypeptides of the invention also include polypeptides that are substantially equivalent to the polypeptides of the invention. In some embodiments, polypeptides according to the invention have at least about 80%, or at least about 90%, or at least about 95%, sequence identity to a polypeptide of the invention. In some embodiments, the invention also includes polypeptides that have homology of at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7% identity with the sequence of the polypeptides in Table 1 which are encoded by SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, and 77-96, or fragments thereof.

In some embodiments, amino acid “substitutions” for creating variants are preferably the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

In some embodiments, substitutions are limited to substitutions in amino acids not conserved among other proteins which have similar identified enzymatic activity. These equivalent amino acids can be determined either by depending on their structural homology with the amino acids which they substitute, or on results of comparative tests of biological activity between the different polypeptides, which are capable of being carried out.

The present invention likewise relates to isolated and/or purified nucleotide sequences, characterized in that they are selected from: a) a nucleotide sequence of one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, or 77-96, or one of their fragments; b) a nucleotide sequence homologous to a nucleotide sequence such as defined in a); c) a nucleotide sequence complementary to a nucleotide sequence such as defined in a) or b), and a nucleotide sequence of their corresponding RNA; d) a nucleotide sequence capable of hybridizing under stringent conditions with a sequence such as defined in a), b) or c); e) a nucleotide sequence comprising a sequence such as defined in a), b), c) or d); and f) a nucleotide sequence modified by a nucleotide sequence such as defined in a), b), c), d) or e).

In some embodiments, it may be desirable to modify the polypeptides of the present invention. One of skill will recognize many ways of generating alterations in a given nucleic acid construct to generate variant polypeptides. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see, e.g., Gillam and Smith, Gene 8:81-97, 1979; Roberts et al., Nature 328:731-734, 1987, both of which are incorporated by reference in their entirety for all purposes).

Nucleic acids which encode protein analogs or variants in accordance with this invention (i.e., wherein one or more amino acids are designed to differ from the wild type polypeptide) may be produced using site directed mutagenesis or PCR amplification in which the primer(s) have the desired point mutations. For a detailed description of suitable mutagenesis techniques, see Sambrook et al., Molecular Cloning: A Laboratory Manual, Cold Spring Harbor Laboratory Press, Cold Spring Harbor, N.Y. (1989) and/or Current Protocols in Molecular Biology, Ausubel et al., eds, Green Publishers Inc. and Wiley and Sons, N.Y (1994), each of which is incorporated by reference in its entirety for all purposes. Chemical synthesis using methods well known in the art, such as that described by Engels et al., Angew Chem Intl Ed. 28:716-34, 1989 (which is incorporated by reference in its entirety for all purposes), may also be used to prepare such nucleic acids. In some embodiments, the recombinant nucleic acids encoding the polypeptides of the invention are modified to provide preferred codons which enhance translation of the nucleic acid in a selected organism.

A number of exemplary methods have been developed for the mutagenesis and diversification of polynucleotides encoding polypeptides to target desired properties of specific polypeptides. Such methods are well known to those skilled in the art. Any of these can be used to alter and/or optimize the activity of a lignin utilization polypeptide of the invention. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol. 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

The polynucleotides of the invention also include polynucleotides including nucleotide sequences that are substantially equivalent to the polynucleotides of the invention. Polynucleotides according to the invention can have at least about 80%, more typically at least about 90%, and even more typically at least about 95%, sequence identity to a polynucleotide of the invention. The invention also provides the complement of the polynucleotides including a nucleotide sequence that has at least about 80%, more typically at least about 90%, and even more typically at least about 95%, sequence identity to a polynucleotide encoding a polypeptide recited above. The invention also includes polynucleotides that have homology of at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7% identity with the sequence SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, or 77-96, or fragments thereof. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities.

### Nucleic Acids

In some embodiments, the present invention relates to the nucleic acids that encode, at least in part, the individual peptides, polypeptides, proteins, and groups of polypeptides of the present invention. In some embodiments, the nucleic acids may be natural, synthetic or a combination thereof. The nucleic acids of the invention may be RNA, mRNA, DNA or cDNA.

In some embodiments, the nucleic acids of the invention also include expression vectors, such as plasmids, or viral vectors, or linear vectors, or vectors that integrate into chromosomal DNA. Expression vectors can contain a nucleic acid sequence that enables the vector to replicate in one or more selected host cells. Such sequences are well known for a variety of cells. E.g., the origin of replication from the plasmid pBR322 is suitable for most Gram-negative bacteria. In eukaryotic host cells, e.g., mammalian cells, the expression vector can be integrated into the host cell chromosome and then the vector replicates with the host chromosome. Similarly, vectors can be integrated into the chromosome of prokaryotic cells.

In general, expression vectors containing replicon and control sequences that are derived from species compatible with the host cell are used in connection with a suitable host cell. The expression vector ordinarily carries a replication site, as well as marking sequences that are capable of providing phenotypic selection in transformed cells. For example, E. coli is typically transformed using pBR322, a plasmid derived from an E. coli species (see, e.g., Bolivar et al., (1977) Gene, 2: 95). pBR322 contains genes for ampicillin and tetracycline resistance and thus provides easy means for identifying transformed cells.

Expression vectors also generally contain a selection gene, also termed a selectable marker. Selectable markers are well-known in the art for prokaryotic and eukaryotic cells, including host cells of the invention. Generally, the selection gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. In some embodiments, an exemplary selection scheme utilizes a drug to arrest growth of a host cell. Those cells that are successfully transformed with a heterologous gene produce a protein conferring drug resistance and thus survive the selection regimen. Other selectable markers for use in bacterial or eukaryotic (including mammalian) systems are well-known in the art.

The expression vector for producing the polypeptides of the invention contain a suitable control region that is recognized by the host organism and is operably linked to the nucleic acid encoding the polypeptide of interest. Promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

Promoters suitable for use with prokaryotic hosts include the beta-lactamase and lactose promoter systems (Chang et al., (1978) Nature, 275: 615; Goeddel et al., (1979) Nature, 281: 544), the arabinose promoter system (Guzman et al., (1992) J. Bacteriol., 174: 7716-7728), alkaline phosphatase, a tryptophan (trp) promoter system (Goeddel, (1980) Nucleic Acids Res., 8: 4057 and EP 36,776) and hybrid promoters such as the tac promoter (deBoer et al., (1983) Proc. Natl. Acad. Sci. USA, 80: 21-25). Other exemplary bacterial promoters include lad, lacZ, T3, T7, gpt, lambda PR, and PL. Other bacterial promoters suitable for expression vectors are also well known in the art. Exemplary eukaryotic promoters include CMV immediate early, HSV thymidine kinase, early and late SV40, LTRs from retrovirus, and mouse metallothionein I. The nucleotide sequences of these and many other promoters have been published, thereby enabling a skilled worker operably to ligate them to DNA encoding the polypeptide of interest (Siebenlist et al, (1980) Cell, 20: 269) using linkers or adaptors to supply any required restriction sites. See also, e.g., Sambrook et al., Molecular Cloning: A Laboratory Manual, Cold Spring Harbor Laboratory Press, Cold Spring Harbor, N.Y. (1989); and Current Protocols in Molecular Biology, Ausubel et al., eds, Green Publishers Inc. and Wiley and Sons, N.Y (1994), both of which are incorporated by reference in their entirety for all purposes.

Control regions for use in bacterial systems also generally contain a Shine-Dalgarno (S.D.) sequence operably linked to the DNA encoding the polypeptide of interest. The Shine-Dalgarno sequence and the initiating ATG codon are used in the initiation of translation by the ribosome in bacterial systems.

Expression vectors of the invention typically have promoter elements, e.g., enhancers, to regulate the frequency of transcriptional initiation. Typically, these are located in the region 30-110 base pairs upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 base pairs apart before activity begins to decline. Depending on the promoter, it appears that individual elements can function either cooperatively or independently to activate transcription.

The present invention also provides nucleic acids that encode the polypeptides of the invention. The nucleic acid encoding a polypeptide of the invention can be easily prepared from an amino acid sequence of the polypeptide of interest using the genetic code. The nucleic acid encoding a polypeptide of the present invention can be prepared using a standard molecular biological and/or chemical procedure. For example, based on the base sequence, a nucleic acid can be synthesized, and the nucleic acid of the present invention can be prepared by combining DNA fragments which are obtained from a cell or other nucleic acid using a polymerase chain reaction (PCR).

The nucleic acid of the present invention can be linked to another nucleic acid so as to be expressed under control of a suitable promoter. The nucleic acid of the present invention can be also linked to, in order to attain efficient transcription of the nucleic acid, other regulatory elements that cooperate with a promoter or a transcription initiation site, for example, a nucleic acid comprising an enhancer sequence, or a terminator sequence. In addition to the nucleic acid of the present invention, a gene that can be a marker for confirming expression of the nucleic acid (e.g. a drug resistance gene, a gene encoding a reporter enzyme, or a gene encoding a fluorescent protein) may be incorporated.

When the nucleic acid of the present invention is introduced into a host cell, the nucleic acid of the present invention may be combined with a substance that promotes transference of a nucleic acid into a cell, for example, a reagent for introducing a nucleic acid such as a liposome or a cationic lipid, in addition to the aforementioned excipients. Alternatively, a vector carrying the nucleic acid of the present invention is also useful.

### Host Cells

In the present invention, various host cells can be used with the polynucleotides and polypeptides of the invention. The host cell may be any of the host cells familiar to those skilled in the art, including prokaryotic cells, eukaryotic cells, such as bacterial cells, fungal cells, yeast cells, mammalian cells, insect cells, or plant cells. Suitable prokaryotic host cells for expression of the polypeptide of the invention are well known in the art. Suitable prokaryote host cells include bacteria, e.g., eubacteria, such as Gram-negative or Gram-positive organisms, for example, any species of Acidovorax, Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas, including, e.g., E. coli, B. subtilis, P. aeruginosa, Salmonella typhimurium, Bacillus cereus, Pseudomonas fluorescens, Serratia marcescens, Clostridium acetobutylicum, Clostridium Beijerinckii, Clostridium saccharoperbutylacetonicum, Clostridium saccharobutylicum, Clostridium aurantibutyricum, or Clostridium tetanomorphum.

One example of an E. coli host is E. coli 294 (ATCC 31,446). Other strains such as E. coli B, E. coli X1776 (ATCC 31,537), and E. coli W3110 (ATCC 27,325) are also suitable. These examples are illustrative rather than limiting. Strain W3110 is a typical host because it is a common host strain for recombinant DNA product fermentations. In one aspect of the invention, the host cell should secrete minimal amounts of proteolytic enzymes. For example, strain W3110 may be modified to effect a genetic mutation in the genes encoding proteins, with examples of such hosts including E. coli W3110 strains 1A2, 27A7, 27B4, and 27C7 described in U.S. Pat. No. 5,410,026 issued Apr. 25, 1995, which is incorporated by reference in its entirety for all purposes.

In some embodiments the host cells are plant cells. In some embodiments the plant cells are cells of monocotyledonous or dicotyledonous plants, including, but not limited to, alfalfa, almonds, asparagus, avocado, banana, barley, bean, blackberry, brassicas, broccoli, cabbage, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis, woody plants such as coniferous and deciduous trees, an ornamental plant, a perennial grass, a forage crop, flowers, other vegetables, other fruits, other agricultural crops, herbs, grass, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.). The term “plants” refers to all physical parts of a plant, including seeds, seedlings, saplings, roots, tubers, stems, stalks, foliage and fruits.

In other embodiments, the host cells are algal and/or photosynthetic, including but not limited to algae or photosynthetic cells of the genera Agmenellum, Amphora, Anabaena, Ankistrodesmus, Botryococcus, Boekelovia, Borodinella, Botryococcus, Carteria, Chaetoceros, Chlamydomonas, Chlorella, Chlorococcum, Chlorogonium, Chrysosphaera, Cricosphaera, Cryptomonas, Cyclotella, Dunaliella, Ellipsoidon, Eremosphaera, Euglena, Fragilaria, Gleocapsa, Gloeothamnion, Hymenomonas, Isochrysis, Lepocinclis, Monoraphidium, Nannochloris, Nannochloropsis, Navicula, Nephrochloris, Nitschia, Nitzschia, Ochromonas, Oocystis, Oscillatoria, Nitzschia, Pascheria, Phagus, Phormidium, Platymonas, Pleurochrysis Prototheca, Pyrobotrys Scenedesmus, Spirogyra, Tetraedron, Tetraselmis, or Volvox. In some embodiments, the host cell is Botryococcus braunii, Prototheca krugani, Prototheca moriformis, Prototheca portoricensis, Prototheca stagnora, Prototheca wickerhamii, or Prototheca zopfii.

In some embodiments, the eukaryotic cells are fungi cells, including, but not limited to, fungi of the genera Aspergillus, Candida, Chlamydomonas, Chrysosporium, Cryotococcus, Debaromyces, Fusarium, Hansenula, Kluyveromyces, Neotyphodium, Neurospora, Penicillium, Pichia, Saccharomyces, Schizosaccharomyce, Trichoderma, Xanthophyllomyces, Yarrowia, and Zygosaccharomyces. Exemplary fungi cells include Saccharomyces cerevisiae, Saccharomyces carlsbergensis, Saccharomyces lactis, Schizosaccharomyces pompe, Kluyveromyces lactis, Pichia pastoris, Hansenula polymorpha, or filamentous fungi, e.g. Trichoderma, Aspergillus sp., including Aspergillus niger, Aspergillus phoenicis, Aspergillus carbonarius.

Exemplary insect cells include any species of Spodoptera or Drosophila, including Drosophila S2 and Spodoptera Sf9. Exemplary animal cells include CHO, COS or Bowes melanoma or any appropriate mouse or human cell line known to person of skill in the art.

### Introduction of Polynucleotides to Host Cells

In some embodiments, the nucleic acids encoding the lignin utilizing polypeptides of the present invention is/are inserted into a vector(s), and the vector(s) is introduced into a cell. In some embodiments, the nucleic acid(s) encoding the lignin utilizing polypeptides is/are introduced to the eukaryotic cell by transfection (e.g., Gorman, et al. Proc. Natl. Acad. Sci. 79.22 (1982): 6777-6781; which is incorporated by reference in its entirety for all purposes), transduction (e.g., Cepko and Pear (2001) Current Protocols in Molecular Biology unit 9.9; DOI: 10.1002/0471142727.mb0909s36, which is incorporated by reference in its entirety for all purposes), calcium phosphate transformation (e.g., Kingston, Chen and Okayama (2001) Current Protocols in Molecular Biology Appendix 1C; DOI: 10.1002/0471142301.nsa01cs01, which is incorporated by reference in its entirety for all purposes), cell-penetrating peptides (e.g., Copolovici, Langel, Eriste, and Langel (2014) ACS Nano 2014 8 (3), 1972-1994; DOI: 10.1021/nn4057269, which is incorporated by reference in its entirety for all purposes), electroporation (e.g Potter (2001) Current Protocols in Molecular Biology unit 10.15; DOI: 10.1002/0471142735.im1015s03 and Kim et al (2014) Genome 1012-19. doi:10.1101/gr.171322.113, Kim et al. 2014 describe the Amaza Nucleofector, an optimized electroporation system, both of these references are incorporated by reference in their entirety for all purposes), microinjection (e.g., McNeil (2001) Current Protocols in Cell Biology unit 20.1; DOI: 10.1002/0471143030.cb2001s18, which is incorporated by reference in its entirety for all purposes), liposome or cell fusion (e.g., Hawley-Nelson and Ciccarone (2001) Current Protocols in Neuroscience Appendix 1F; DOI: 10.1002/0471142301.nsa01fs10, which is incorporated by reference in its entirety for all purposes), mechanical manipulation (e.g. Sharon et al. (2013) PNAS 2013 110(6); DOI: 10.1073/pnas.1218705110, which is incorporated by reference in its entirety for all purposes) or other well-known technique for delivery of nucleic acids to eukaryotic cells. Once introduced, the nucleic acids of the invention can be transiently expressed episomally, or can be integrated into the genome of the host cell using well known techniques such as recombination (e.g., Lisby and Rothstein (2015) Cold Spring Harb Perspect Biol. March 2; 7(3). pii: a016535. doi: 10.1101/cshperspect.a016535, which is incorporated by reference in its entirety for all purposes), or non-homologous integration (e.g., Deyle and Russell (2009) Curr Opin Mol Ther. 2009 August; 11(4):442-7, which is incorporated by reference in its entirety for all purposes). The efficiency of homologous and non-homologous recombination can be facilitated by genome editing technologies that introduce targeted double-stranded breaks (DSB). Examples of DSB-generating technologies are CRISPR/Cas9, TALEN, Zinc-Finger Nuclease, or equivalent systems (e.g., Cong et al. Science 339.6121 (2013): 819-823, Li et al. Nucl. Acids Res (2011): gkr188, Gajet al. Trends in Biotechnology 31.7 (2013): 39.7-405, all of which are incorporated by reference in their entirety for all purposes), transposons such as Sleeping Beauty (e.g., Singh et al (2014) Immunol Rev. 2014 January; 257(1):181-90. doi: 10.1111/imr.12137, which is incorporated by reference in its entirety for all purposes), targeted recombination using, for example, FLP recombinase (e.g., O'Gorman, Fox and Wahl Science (1991) 15:251(4999):1351-1355, which is incorporated by reference in its entirety for all purposes), CRE-LOX (e.g., Sauer and Henderson PNAS (1988): 85; 5166-5170), or equivalent systems, or other techniques known in the art for integrating the nucleic acids of the invention into the eukaryotic cell genome.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Techniques for transforming a wide variety of higher plant species are well known and described in the technical and scientific literature. See, e.g., Weising (1988) Ann. Rev. Genet. 22:421-477; U.S. Pat. No. 5,750,870, which are both incorporated by reference in their entirety for all purposes.

### Uses of Polynucleotides, Polypeptides and Extracts

In some embodiments, the polypeptides and/or polynucleotides of the invention are used in industrial processes in a variety of forms, including cell-based systems and/or as partially or substantially purified forms, or in mixtures or other formulations. In one aspect, commercial (e.g., “upscaled”) enzyme production systems are used, and this invention can use any polypeptide production system known in the art, including any cell-based expression system, which include numerous strains, including any eukaryotic or prokaryotic system, including any insect, microbial, yeast, bacterial and/or fungal expression system; these alternative expression systems are well known and discussed in the literature and all are contemplated for commercial use for producing and using the enzymes of the invention. For example, Bacillus species can be used for industrial production (see, e.g., Canadian Journal of Microbiology, 2004 January, 50(1):1-17, which is incorporated by reference in its entirety for all purposes). Alternatively, Streptomyces species, such as S. lividans, S. coelicolor, S. limosus, S. rimosus, S. roseosporus, and S. lividans can be used for industrial and sustainable production hosts (see, e.g., Appl Environ Microbiol. 2006 August; 72(8): 5283-5288, which is incorporated by reference in its entirety for all purposes). Any Fusarium sp. can be used in an expression system to practice this invention, including e.g., Fusarium graminearum; see e.g., Royer et al. Bio/Technology 13:1479-1483 (1995), which is incorporated by reference in its entirety for all purposes. Any Aspergillus sp. can be used in an expression system to practice this invention, including e.g., A. nidulans; A. fumigatus; Aspergillus phoenicis, A. niger, A. carbonarius, or A. oryzae; the genome for A. niger CB S513.88, a parent of commercially used enzyme production strains, was recently sequenced (see, e.g., Nat Biotechnol. 2007 February; 25(2):221-31; World Journal of Microbiology and Biotechnology, 2001, 17(5):455-461, both of which are incorporated by reference in their entirety for all purposes). Similarly, the genomic sequencing of Aspergillus oryzae was recently completed (Nature. 2005 Dec. 22; 438(7071):1157-61, which is incorporated by reference in its entirety for all purposes). For alternative fungal expression systems that can be used to practice this invention, e.g., to express enzymes for use in industrial applications, such as biofuel production, see e.g., Advances in Fungal Biotechnology for Industry, Agriculture, and Medicine. Edited by Jan S. Tkacz & Lene Lange. 2004. Kluwer Academic & Plenum Publishers, New York; and e.g., Handbook of Industrial Mycology. Edited by Zhiqiang An. 24 Sep. 2004. Mycology Series No. 22. Marcel Dekker, New York; and e.g., Talbot (2007) “Fungal genomics goes industrial”, Nature Biotechnology 25(5):542; and in U.S. Pat. Nos. 4,885,249; 5,866,406; and international patent publication WO/2003/012071, all of which are incorporated by reference in their entirety for all purposes.

The invention provides a method for expressing recombinant lignin utilizing polypeptides, e.g., the polypeptides of SEQ ID NOS: 2, 4, 6, 8, 10, 12, 14, 16, 18, 1-20, 22, 24, 26, 28, 30, 32, 34, 36, 38, 40, 42, 44, 46, 48, 50, 52, 54, 56, 58, 60, 62, 64, 66, 68, 70, 72, 74, or 76, and the sets of polypeptides in Table 3 which are encoded by SEQ ID NOS: 77-96 in a cell comprising expressing the polypeptides in a nucleic acid of the invention, e.g., a nucleic acid comprising a nucleic acid sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, or 77-96 or an exemplary sequence of the invention over a region of at least about 100 residues, wherein the sequence identities are determined by analysis with a sequence comparison algorithm or by visual inspection, or, a nucleic acid that hybridizes under stringent conditions to a nucleic acid sequence of the invention. The expression can be effected by any means, including e.g., use of a high activity promoter, a dicistronic vector or by gene amplification of the vector.

Cells can be harvested by centrifugation, disrupted by physical or chemical means and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

In some embodiments, polypeptides and methods for utilization of lignin are used in polypeptide ensembles (“mixtures” or “cocktails”) for the efficient hydrolysis (e.g., depolymerization) of lignin to metabolizeable carbon moieties, including sugars, alcohols, other molecules of intermediate metabolism, and/or other precursor chemicals. Exemplary polypeptide cocktails are described herein; however, the invention encompasses compositions comprising mixtures of polypeptides comprising at least one polypeptide of the invention; and in some embodiments, a mixture (“ensembles” or “cocktails”) of the invention can also comprise any other polypeptide of the invention, and the like. As discussed above, the invention provides methods for discovering and implementing the most effective combination of polypeptides of the invention to enable new “biomass conversion”, “biomass processing”, alternative energy, biofuel production, and/or industrial processes.

In some embodiments, nucleic acids and polypeptides of the invention having lignin utilizing activity are used in processes for converting lignin biomass to sugar and precursor molecules, which are converted by methods well-known in the art to many products, including for example, biofuels, bioalcohols, synthetic fibers, plastics, rubber, oleochemicals, foods, cosmetics, polymer products, etc. In some embodiments, the sugars made by the methods of the invention include, for example, glucose, galactose, sucrose, fructose, etc. In some embodiments, the methods of the invention produce a bioalcohol such as, for example, biomethanol, bioethanol, biopropanol, bioisopropanol, biobutanol, biopentanol, biodiols (such as propane diols, butane diols, pentane diols, etc.) from compositions comprising lignin biomass. In some embodiments, the methods of the invention produce alkanes, alkenes, dialkenes, or alkynes such as, for example, propene, butene, butadiene, pentene, pentadiene, etc. In some embodiments, the methods of the invention produce oleochemicals such as surfactants, detergents, soaps, cosmetics, lubricants, etc. In some embodiments, the methods of the invention produce foods such as sugars, flours, protein supplements, etc. In some embodiments, the methods of the invention produce biofuels such as, for example, biodiesel, biojet fuel, bioalcohols as fuel additives (e.g., bioethanol), biofuel gasoline, biogas, syngas, bioethers, etc.

The lignin biomass material can be obtained from herbaceous and woody energy crops, as well as agricultural crops, i.e., the plant parts, primarily stalks and leaves, not removed from the fields with the primary food or fiber product. Examples include agricultural wastes such as sugarcane bagasse, rice hulls, corn fiber (including stalks, leaves, husks, and cobs), wheat straw, rice straw, sugar beet pulp, citrus pulp, citrus peels; forestry wastes such as hardwood and softwood thinnings, and hardwood and softwood residues from timber operations; wood wastes such as saw mill wastes (wood chips, sawdust) and pulp mill waste; urban wastes such as paper fractions of municipal solid waste, urban wood waste and urban green waste such as municipal grass clippings; and wood construction waste. Additional lignin biomass materials include dedicated cellulosic crops such as switchgrass, hybrid poplar wood, and miscanthus, fiber cane, and fiber sorghum. Five-carbon sugars that are produced from such materials include xylose.

Examples of paper or wood waste suitable for treatment with polypeptides of the invention include discarded or used photocopy paper, computer printer paper, notebook paper, notepad paper, typewriter paper, and the like, as well as newspapers, magazines, cardboard, and paper-based packaging materials and recycled paper materials. In addition, urban wastes, e.g. the paper fraction of municipal solid waste, municipal wood waste, and municipal green waste, along with other materials containing sugar, starch, and/or cellulose can be used.

The enzymes of the invention used to treat or process the lignin biomass material (e.g., from agricultural crops, food or feed production byproduct, lignin waste products, plant residues, sugarcane bagasse, corn or corn fiber, waste wood or paper, etc.), in addition to being directly added to the material, alternatively can be made by a microorganism (e.g., a virus, plant, yeast, etc.) living on or within the biomass material, or by the biomass material itself, e.g., as a transgenic plant or seed and the like. In some embodiments, microorganisms that produce polypeptides of the invention are added to the biomass material to be processed. These microorganisms can be the sole source of the polypeptide of the invention, or can supplement a cocktail that has polypeptides of the invention in another form (e.g., as either a purified enzyme, or in crude lysate of a culture, such as a bacterial, yeast or insect cell culture, or any other formulation), or to supplement the presence of the polypeptide of the invention as a heterologous recombinant protein in a transgenic plant. In some embodiments, the plant can be engineered to express the enzyme recombinantly by transient infection, transformation or transduction with naked DNA, plasmid, virus and the like. In some embodiments, the enzymes are produced in plants or plant seeds, like corn, and then the enzyme can be isolated from the plant or the plant can be used directly in the process. In some embodiments, the polypeptides of the invention can be added to the treatment process in batches, by fed-batch processes, added continually and/or be recycled during the process. In some embodiments, the cells, polypeptides, and/or extracts of the invention increase utilization of a biomass by separating other components of the biomass (e.g., cellulose) from lignin. In some embodiments, the lignin is chemically transformed into smaller components and this process allows other components in the biomass to be utilized more efficiently and/or more completely. In some embodiments, the lignin is bound or sequestered by polypeptides of the invention and this allows other components of the biomass to be utilized more efficiently and/or more completely.

The polypeptides described in this invention can be used in the form of a cell extracts and/or supernatants from host organisms. These extracts can further be supplemented with additional extracts, purified proteins or small molecules such as oxidative mediators. Extracts have been used to modify a heterogeneous aromatic polymer. For example, the extracts have been used to modify a heterogeneous aromatic polymer such as lignin in lignocellulose. The modifications have been shown to improve utilization of lignin by decreasing total protein loading, including the amount of cellulases, or boosting glucose yields. In some embodiments, the extracts act on a heterogeneous polymer such as lignin or coal to release aromatic chemicals. In other embodiments, the extracts act on a heterogeneous polymers such as lignin or coal to change properties of the substrates for the production of fibers, resins or materials.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### Examples

### Example 1

### Functional Metagenomic Library Screening for Lignin Utilization

Metagenomic libraries from CO182 and CO183 were constructed using the Fosmid Copy Control system (pCC1FOS) from EpiCentre, as previous reports suggest that increased copy number enhances heterologous gene expression in the EPI300 E. coli host. Martinez, A., Bradley, A. S., Waldbauer, J. R., Summons, R. E. & Delong, E. F. Proteorhodopsin photosystem gene expression enables photophosphorylation in a heterologous host. PNAS 104, 5590-5595 (2007), which is incorporated by reference in its entirety for all purposes. A total of 46,000 fosmids arrayed in 384-well plates were grown in the presence of HKL-F1 overnight prior to the addition of the biosensor. These metagenomics libraries were functionalized by the addition of the PemrR-GFP biosensor (a reporter strain) which was transferred to the pCC1FOS vector used in library production to facilitate co-culture based screening using shared antibiotic selection. A metagenomics analysis of hydrocarbon resource environments indicates aerobic taxa and genes to be unexpectedly common. D. et al. Environ. Sci. Technol. DOI: 10.1021/es4020184 (2013), which is incorporated by reference in its entirety for all purposes.

Co-cultures were subsequently grown for three hours prior to measuring GFP fluorescence. Fluorescent signals were normalized to background and corrected for edge effects. Consequently, 24 fosmids activating the emrR biosensor (16 from CO182 and 8 from CO183) were selected for downstream functional characterization and sequencing.

### Example 2

### Lignin Transformation Testing of Fosmids

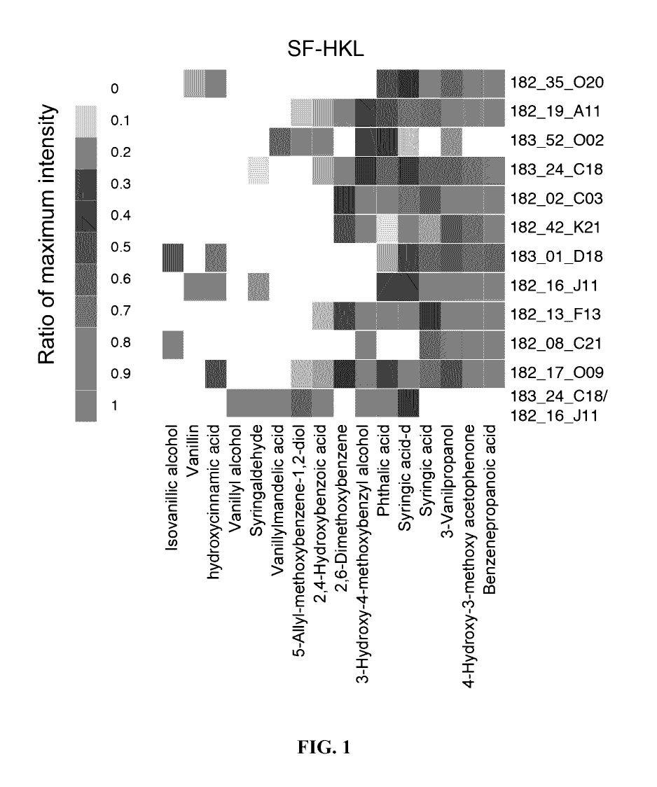

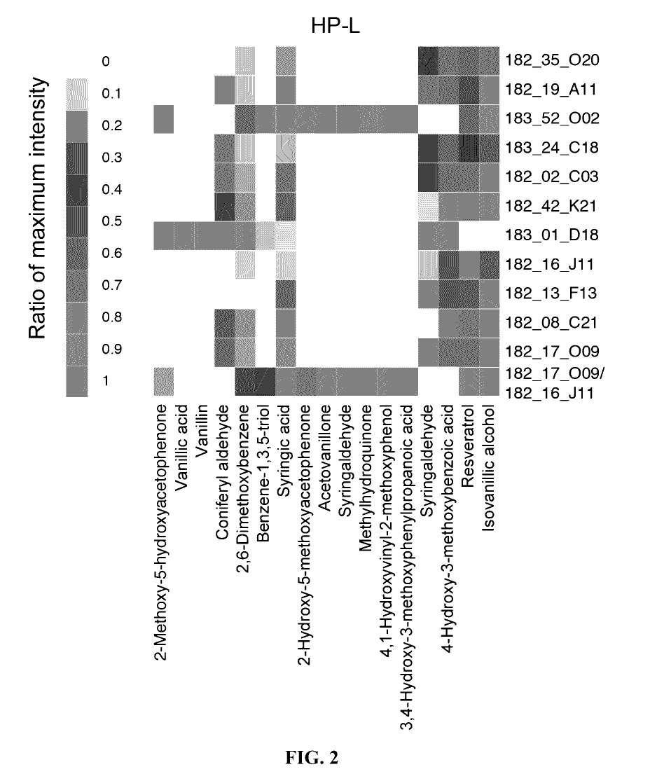

To verify the production of lignin transformation products by fosmids activating the PemrR-GFP biosensor, 11 of the most active clones were incubated in the presence of HKL-F1 and a second industrially purified high-performance lignin (HP-L™) substrate. Arato, C., Pye, E. K. and Gjennestad, G. The lignol approach to biorefining of woody biomass to produce ethanol and chemicals, Appl Biochem Biotech. 123, 871-882 (2005), which is incorporated by reference in its entirety for all purposes. Lignin transformation products including acetovanillone, 5-allyl-methoxybenzene-1,2-diol, benzenepropanoic acid, benzene-1,3,5-triol, coniferyl aldehyde, 3,5-dimethyl-4-hydroxycinnamic acid, 2,6-dimethoxybenzene, 2,4-hydroxybenzoic acid, 4-hydroxy-3-methoxy acetophenone, 2-hydroxy-5-methoxy acetophenone, 4-hydroxy-3-methoxybenzoic acid, 3-hydroxy-4-methoxybenzyl alcohol, isovanillic alcohol, 2-methoxy-5-hydroacetophenone, phthalic acid, resveratrol, syringaldehyde, syringaldehyde, syringic acid-d, syringic acid, vanillin, vanillic acid, vanillyl alcohol, vanillylmandelic acid, and 3-vanilpropanol were then measured by gas chromatography-mass spectrometry (GC-MS). An array of monoaromatic compound profiles were observed for single fosmid incubations, which varied between HKL-F1 and HP-L™ as consistent with different substrate properties or varying specificities of fosmid encoded enzymes (FIGS. 1 and 2). Fosmid co-cultures exhibited synergy in combination, producing monoaromatic compound profiles that differed from individual fosmid incubation profiles in unexpected ways (FIGS. 1 and 2). Moreover, while single fosmid incubations with HKL-F1 led to precipitate formation, only co-culture fosmid incubations were capable of forming precipitates with HP-L™.

The observations confirm that fosmids recovered in the PemrR-GFP biosensor screen confer lignin transformation phenotypes with different end product profiles.

### Example 3

### Gene Analysis

Random transposon mutagenesis identified genes encoded on the 11 characterized fosmids necessary for activating the PemrR-GFP biosensor. Nine out of 11 fosmids contained transposon insertions capable of reducing biosensor activation in two or more genes, suggesting that the observed lignin transforming phenotypes require multiple pathway components.

Consistent with this observation, mapping the location of each transposon insertion identified six functional classes implicated in lignin transformation. These included genes predicted to encode electron transfer (unassigned oxidoreductase activity), co-factor generation (hydrogen peroxide formation), protein secretion (secretion apparatus or signal peptide), small molecule transport (multidrug efflux superfamily), motility (methyl accepting chemotaxis proteins (MCP)), and signal transduction (PAS domain containing sensors) pathway components. Full-fosmid sequencing and comparative analysis of all 24 fosmids activating the PemrR-GFP biosensor also identified recurring subsets of genes on typically non-syntenic clones encoding one or more of the six functional classes identified by transposon mutagenesis.

While electron transfer, co-factor generation and protein secretion have well-defined roles in lignin transformation, the roles of the remaining three functional groups are novel. It is notable that several of the fosmids identified with the PemrR-GFP biosensor actually encode small molecular transport systems similar to emrR and emrB, further reinforcing a role for these genes in regulating microbial responses to monoaromatic exposure in the environment (see TABLES 4 and 5). Cell motility could play a role in establishing optimal cell positioning along transformational gradients.

TABLE 4
SF-HKL

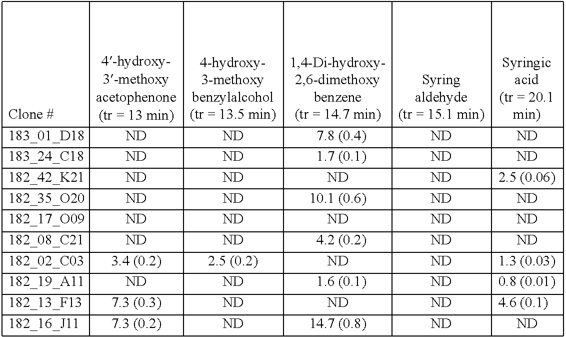

TABLE 5
HP-L

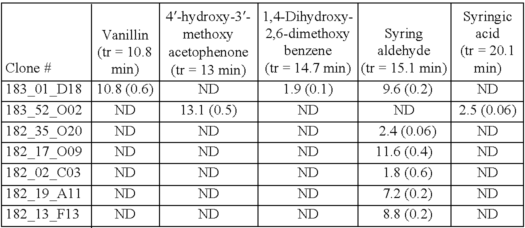

This relationship between lignin transformation and cell motility is highlighted by a recent study that observed an enrichment of motility related genes and transcripts in wood feeding termites relative to dung-feeding termites. He, S. et al. Comparative metagenomic and metatransciptomic analysis of hindgut paunch microbiota in wood and dung feeding higher termites. He et al., PLoS ONE. 8(4): e61126 (2013), which is incorporated by reference in its entirety for all purposes.

Finally, signal transducers could play a role in mediating lignin substrate specificity among and between microbial groups and contribute to gradient formation. The necessity of genes encoding both MCP and signal transduction on the fosmids identified in this study directly implicates both of these functional classes in mediating lignin transformation phenotypes in the environment.

In addition to the six functional classes described above, 16 of the 24 fully sequenced fosmids harbored mobile genetic elements (MGE). These elements were typically located proximal to one or more of the six functional classes suggesting a role for metabolic island or islet formation in propagating lignin transformation phenotypes in the environment. To further explore the relationship between lignin transformation phenotypes and genomic island or islet formation coverage depth, G+C content variation and tRNA positioning on the active fosmids was examined. Fragment recruitment of 500 million unassembled Illumina reads sourced from CO182 and CO183 environmental DNA identified abrupt changes in coverage depth in genomic intervals harboring MGE and one or more of the six functional classes consistent with island formation. The presence of islands was further supportedin 8 of the fosmids where coverage changes were associated with variation in median G+C composition or tRNA gene positioning. Tables 1 and 3 list exemplary nucleic acids identified and isolated with positive fosmids.

Two transposon mutants (i.e. position 4949 and position 55060) of fosmid 182_08_C21 (Annotation No. KJ802937) show a reduction in lignin utilization as demonstrated by a reduction in intermediates formed during lignin utilization. These mutants correspond to SEQ ID NOS: 1 and 13, which are both members of the oxidoreductase gene group.

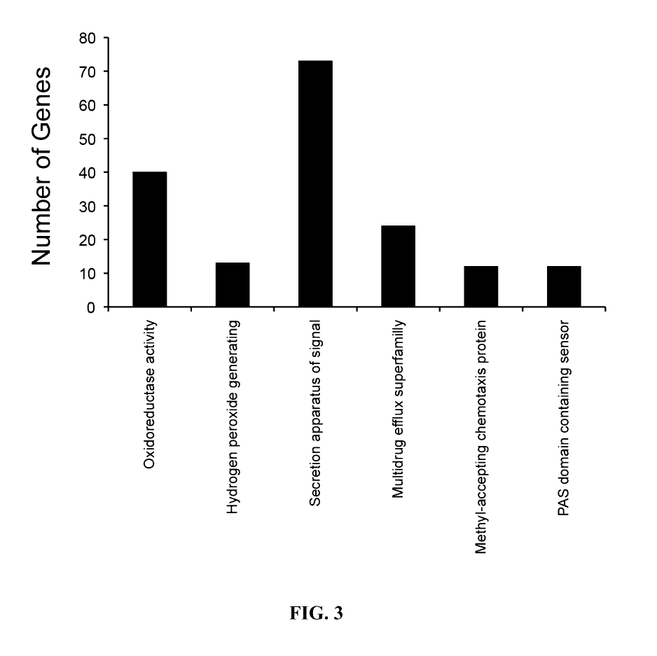

FIG. 3 provides a graphical representation of the relative proportions of genes grouped into the six functional classes, implicated in lignin utilization phenotypes (out of 813 total genes) in the active fosmids identified in the exemplary screen. Example 4. Host Cell Extracts for Lignin Utilization

Fosmid ID nos. 182_35_O20 (Annotation No. KJ802947), 182_16_J11 (Ann. No. KJ802944), 182_11_B22 (Ann. No. KJ802940), 182_09_J11 (Ann. No. KJ802938), 182_42_K21 (Ann. No. KJ802948), 182_02_CO3 (Ann. No. KJ802934), or 182_16_E12 (Ann. No. KJ802943) were placed in E. coli. Extracts were prepared from these E. coli by chemical means that include detergents such as SDS. The extracts were subsequently added to a 10 Da filter apparatus for concentration and buffer exchange.

The extracts were used with a biomass obtained from poplar. Steam treated poplar was mixed with extracts to give 10 mg of soluble protein (from the extract) per gram of biomass. The hydrolysis reaction was performed at 50° C. for 48 hrs and the reaction conditions comprised 50 mM Na-acetate (pH5), 1 mM MnSO4, and 5% (w/w) substrate. The E. coli extracts from fosmids 182_35_020 (Annotation No. KJ802947), 182_16_J11 (Ann. No. KJ802944), 182_11_B22 (Ann. No. KJ802940), 182_09_J11 (Ann. No. KJ802938), 182_42_K21 (Ann. No. KJ802948), 182_02_CO3 (Ann. No. KJ802934), or 182_16_E12 (Ann. No. KJ802943) showed increased utilization of the biomass.

### Example 5

### Extracts from E. coli Containing SEQ ID NO 75

A vector suitable for use in E. coli is engineered to contain SEQ ID NO. 75, nucleotides 169-6997 from fosmid 182_35_O20. The vector with SEQ ID NO 75 is placed into E. coli using standard techniques. E. coli with the vector is grown overnight in LB, the E. coli cells are recovered, and then exposed to a media containing lignin. The E. coli cells are incubated in the lignin media for 16 hours, and then the cells are isolated. Isolated cells are disrupted by standard methods, and extracts are prepared from the cells.

The extract obtained from the E. coli cells are added to a biomass material containing lignin. The polypeptides in the extract utilize the lignin in the biomass. Optionally, other polypeptides are added to the biomass for digesting the cellulose in the biomass. Including the E. coli extracts increases the utilization of cellulose in the biomass.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

TABLE 2

| | Is- | | | | |
|---|---|---|---|---|---|
| | land | | | | |
| | num- | | Island | Island | |
| Fosmid ID | ber | Accession | start | stop | SEQ ID No |
| 182_2_C03 | 1 | KJ802934 | 1 | 43497 | SEQ ID NO: 77 |
| 182_6_L14 | 1 | KJ802935 | 9671 | 27599 | SEQ ID NO: 78 |
| 182_8_C21 | 1 | KJ802937 | 1118 | 17713 | SEQ ID NO: 79 |
| 182_9_J11 | 1 | KJ802938 | 151 | 22544 | SEQ ID NO: 80 |
| 182_10_L09 | 1 | KJ802939 | 18834 | 30312 | SEQ ID NO: 81 |
| 182_11_B22 | 1 | KJ802940 | 6517 | 31166 | SEQ ID NO: 82 |
| 182_13_F13 | 1 | KJ802942 | 2486 | 21209 | SEQ ID NO: 83 |
| 182_16_E12 | 1 | KJ802943 | 2 | 25718 | SEQ ID NO: 84 |
| 182_16_J11 | 1 | KJ802944 | 182 | 3249 | SEQ ID NO: 85 |
| 182_16_J11 | 2 | KJ802944 | 3749 | 28341 | SEQ ID NO: 86 |
| 182_17_9 | 1 | KJ802945 | 3 | 19794 | SEQ ID NO: 87 |
| 182_19_A11 | 1 | KJ802946 | 4756 | 32797 | SEQ ID NO: 88 |
| 182_35_20 | 1 | KJ802947 | 169 | 6997 | SEQ ID NO: 89 |
| 182_42_K21 | 1 | KJ802948 | 2 | 39464 | SEQ ID NO: 90 |
| 183_1_D18 | 1 | KJ802949 | 27956 | 34694 | SEQ ID NO: 91 |
| 183_1_D18 | 2 | KJ802949 | 1 | 27576 | SEQ ID NO: 92 |
| 183_21_D14 | 1 | KJ802951 | 2 | 35892 | SEQ ID NO: 93 |
| 183_24_C18 | 1 | KJ802952 | 2 | 12903 | SEQ ID NO: 94 |
| 183_29_M04 | 1 | KJ802954 | 9978 | 34744 | SEQ ID NO: 95 |
| 183_52_O2 | 1 | KJ802957 | 1 | 5196 | SEQ ID NO: 96 |

TABLE 3

| Fosmid/Gene | | Island | ORF | ORF | |
|---|---|---|---|---|---|
| ID | Accession | No. | start | stop | Description |
| 182_09_J11_1 | KJ802938 | 1 | 151 | 1440 | putative aminopeptidase 2 |
| 182_09_J11_2 | KJ802938 | 1 | 1513 | 2475 | NAD(P)(H)-dependent |
| | | | | | oxidoreductase |
| 182_09_J11_3 | KJ802938 | 1 | 2649 | 4733 | prc gene product |
| 182_09_J11_4 | KJ802938 | 1 | 4905 | 5372 | TPR repeat, SEL1 subfamily |
| | | | | | protein |
| 182_09_J11_5 | KJ802938 | 1 | 5372 | 5743 | hypothetical protein |
| | | | | | PSTAB_1345 |
| 182_09_J11_6 | KJ802938 | 1 | 5740 | 6066 | Cro/Cl family transcriptional |
| | | | | | regulator |
| 182_09_J11_7 | KJ802938 | 1 | 6223 | 6699 | hypothetical protein |
| | | | | | A458_07510 |
| 182_09_J11_8 | KJ802938 | 1 | 7028 | 7342 | helix-hairpin-helix repeat- |
| | | | | | containing compet |
| 182_09_J11_9 | KJ802938 | 1 | 7463 | 8731 | flagellar hook-associated |
| | | | | | protein FlgL |
| 182_09_J11_10 | KJ802938 | 1 | 8744 | 10759 | flagellar hook-associated |
| | | | | | protein FlgK |
| 182_09_J11_11 | KJ802938 | 1 | 10763 | 11935 | flagellar rod assembly |
| | | | | | protein/muramidase Fl |
| 182_09_J11_12 | KJ802938 | 1 | 11946 | 13046 | flagellar basal body P-ring |
| | | | | | protein |
| 182_09_J11_13 | KJ802938 | 1 | 13061 | 13756 | flagellar basal body L-ring |
| | | | | | protein |
| 182_09_J11_14 | KJ802938 | 1 | 13841 | 14626 | flagellar basal body rod |
| | | | | | protein FlgG |
| 182_09_J11_15 | KJ802938 | 1 | 14662 | 15402 | flagellar basal body rod |
| | | | | | protein FlgF |
| 182_09_J11_16 | KJ802938 | 1 | 15598 | 17184 | flagellar hook protein FlgE |
| 182_09_J11_17 | KJ802938 | 1 | 17214 | 17897 | flagellar basal body rod |
| | | | | | modification protein |
| 182_09_J11_18 | KJ802938 | 1 | 17917 | 18360 | flagellar basal body rod |
| | | | | | protein FlgC |
| 182_09_J11_19 | KJ802938 | 1 | 18372 | 18836 | flagellar basal body rod |
| | | | | | protein FlgB |
| 182_09_J11_20 | KJ802938 | 1 | 18972 | 19796 | chemotaxis protein |
| | | | | | methyltransferase CheR |
| 182_09_J11_21 | KJ802938 | 1 | 19831 | 20763 | chemotaxis protein CheV |
| 182_09_J11_22 | KJ802938 | 1 | 20855 | 21595 | flagellar basal body P-ring |
| | | | | | biosynthesis protein |
| 182_09_J11_23 | KJ802938 | 1 | 21709 | 22038 | negative regulator of flagellin |
| | | | | | synthesis Fl |
| 182_09_J11_24 | KJ802938 | 1 | 22074 | 22544 | FlgN family protein |
| 182_09_J11_25 | KJ802938 | | 22604 | 23350 | type IV pilus assembly PilZ |
| 182_09_J11_26 | KJ802938 | | 23619 | 24830 | phage integrase family site |
| | | | | | specific recombinase |
| 182_09_J11_27 | KJ802938 | | 24827 | 25399 | hypothetical protein |
| | | | | | PMI32_04729 |
| 182_09_J11_28 | KJ802938 | | 25528 | 25728 | excisionase |
| 182_09_J11_30 | KJ802938 | | 25862 | 26206 | hypothetical protein |
| | | | | | G1E_09582 |
| 182_09_J11_31 | KJ802938 | | 26260 | 26727 | virulence-associated protein E |
| 182_09_J11_35 | KJ802938 | | 27828 | 28385 | hypothetical protein |
| | | | | | PfraA_21814 |
| 182_09_J11_36 | KJ802938 | | 29037 | 29210 | hypothetical protein |
| | | | | | PMI22_00482 |
| 182_09_J11_37 | KJ802938 | | 29207 | 29479 | hypothetical protein |
| | | | | | PMI22_00494 |
| 182_09_J11_41 | KJ802938 | | 30075 | 32627 | hypothetical protein |
| 182_09_J11_45 | KJ802938 | | 34092 | 34493 | possible bacteriophage |
| | | | | | terminase small subunit |
| 182_09_J11_46 | KJ802938 | | 35126 | 35752 | resolvase |
| 182_02_C03_1 | KJ802934 | 1 | 1 | 1266 | acyl-CoA dehydrogenase |
| 182_02_C03_2 | KJ802934 | 1 | 1681 | 2328 | peptide methionine sulfoxide |
| | | | | | reductase |
| 182_02_C03_3 | KJ802934 | 1 | 2440 | 5112 | sensory box protein PAS/PAC |
| | | | | | and GAF sensor-containing |
| 182_02_C03_4 | KJ802934 | 1 | 5223 | 5750 | TPR repeat-containing protein |
| 182_02_C03_5 | KJ802934 | 1 | 5838 | 7844 | pyruvate dehydrogenase |
| | | | | | dihydrolipoyltransace |
| 182_02_C03_6 | KJ802934 | 1 | 7872 | 10517 | 2-oxo-acid dehydrogenase E1 |
| | | | | | subunit |
| 182_02_C03_7 | KJ802934 | 1 | 10784 | 13729 | bifunctional glutamine- |
| | | | | | synthetase adenylyltr |
| 182_02_C03_8 | KJ802934 | 1 | 13780 | 14703 | branched-chain amino acid |
| | | | | | aminotransferase |
| 182_02_C03_9 | KJ802934 | 1 | 14778 | 15812 | lipopolysaccharide |
| | | | | | heptosyltransferase II |
| 182_02_C03_10 | KJ802934 | 1 | 15813 | 16814 | lipopolysaccharide |
| | | | | | heptosyltransferase I |
| 182_02_C03_11 | KJ802934 | 1 | 16814 | 17935 | UDP-glucose:(heptosyl) LPS |
| | | | | | alpha 1,3-glucosy |
| 182_02_C03_12 | KJ802934 | 1 | 17979 | 18785 | lipopolysaccharide core |
| | | | | | heptose(I) kinase RfaP |
| 182_02_C03_13 | KJ802934 | 1 | 18785 | 19519 | lipopolysaccharide kinase |
| 182_02_C03_14 | KJ802934 | 1 | 19516 | 20259 | lipopolysaccharide kinase |
| 182_02_C03_15 | KJ802934 | 1 | 20259 | 21704 | serine/threonine protein |
| | | | | | kinase |
| 182_02_C03_16 | KJ802934 | 1 | 21717 | 23471 | carbamoyltransferase |
| 182_02_C03_17 | KJ802934 | 1 | 23458 | 24375 | glycosyl transferase family |
| | | | | | protein |
| 182_02_C03_18 | KJ802934 | 1 | 24390 | 25619 | hypothetical protein |
| | | | | | A458_02260 |
| 182_02_C03_19 | KJ802934 | 1 | 25874 | 26743 | capsule polysaccharide |
| | | | | | biosynthesis |
| 182_02_C03_20 | KJ802934 | 1 | 26740 | 28497 | O-antigen polymerase protein |
| 182_02_C03_21 | KJ802934 | 1 | 28520 | 29122 | toluene tolerance protein |
| 182_02_C03_22 | KJ802934 | 1 | 29158 | 30975 | transport protein MsbA |
| 182_02_C03_23 | KJ802934 | 1 | 30975 | 31871 | Mig-14 family protein |
| 182_02_C03_24 | KJ802934 | 1 | 31875 | 33269 | LmbE family protein |
| 182_02_C03_25 | KJ802934 | 1 | 33347 | 34768 | bifunctional heptose 7- |
| | | | | | phosphate kinase/heptose 1 |
| 182_02_C03_26 | KJ802934 | 1 | 34839 | 35726 | hypothetical protein |
| | | | | | PstZobell_18470 |
| 182_02_C03_27 | KJ802934 | 1 | 35817 | 36626 | aldo/keto reductase family |
| | | | | | oxidoreductase |
| 182_02_C03_28 | KJ802934 | 1 | 36623 | 37798 | oxidoreductase, FAD-binding |
| | | | | | protein |
| 182_02_C03_29 | KJ802934 | 1 | 37859 | 38191 | multidrug efflux SMR |
| | | | | | transporter |
| 182_02_C03_30 | KJ802934 | 1 | 38311 | 39579 | 3-deoxy-D-manno- |
| | | | | | octulosonic-acid transferase |
| 182_02_C03_31 | KJ802934 | 1 | 39753 | 41207 | outer membrane efflux protein |
| | | | | | TolC/Type 1 secretion |
| 182_02_C03_32 | KJ802934 | 1 | 41590 | 43497 | thiamine biosynthesis protein |
| | | | | | ThiC |
| 182_08_C21_1 | KJ802937 | 1 | 1118 | 1465 | site-specific recombinase, |
| | | | | | phage integrase fa |
| 182_08_C21_2 | KJ802937 | 1 | 1684 | 2343 | hypothetical protein |
| | | | | | CLOSCI_03331 |
| 182_08_C21_3 | KJ802937 | 1 | 2693 | 3838 | general secretion pathway |
| | | | | | protein F |
| 182_08_C21_4 | KJ802937 | 1 | 3915 | 5096 | luciferase family |
| | | | | | oxidoreductase |
| 182_08_C21_5 | KJ802937 | 1 | 5112 | 5597 | methyl-accepting chemotaxis |
| | | | | | sensory transduce.. |
| 182_08_C21_6 | KJ802937 | 1 | 5714 | 6478 | response regulator of the |
| | | | | | LytR/AlgR family |
| 182_08_C21_7 | KJ802937 | 1 | 6459 | 7580 | integral membrane sensor |
| | | | | | signal transduction |
| 182_08_C21_8 | KJ802937 | 1 | 7632 | 9089 | argininosuccinate lyase |
| 182_08_C21_9 | KJ802937 | 1 | 9173 | 10225 | catalase |
| 182_08_C21_10 | KJ802937 | 1 | 10420 | 12924 | large extracellular alpha- |
| | | | | | helical protein |
| 182_08_C21_11 | KJ802937 | 1 | 14078 | 14905 | phosphoserine |
| | | | | | aminotransferase |
| 182_08_C21_12 | KJ802937 | 1 | 14898 | 15998 | phosphoserine |
| | | | | | aminotransferase |
| 182_08_C21_13 | KJ802937 | 1 | 15979 | 16482 | hypothetical protein |
| | | | | | YO5_08308 |
| 182_08_C21_14 | KJ802937 | 1 | 16568 | 17713 | pyrroloquinoline quinone |
| | | | | | biosynthesis protei |
| 182_08_C21_15 | KJ802937 | | 17682 | 17978 | pyrroloquinoline quinone |
| | | | | | biosynthesis protei |
| 182_08_C21_16 | KJ802937 | | 18520 | 20040 | aldehyde dehydrogenase |
| 182_08_C21_17 | KJ802937 | | 20285 | 21403 | NADH:flavin oxidoreductase/ |
| | | | | | NADH oxidase |
| 182_08_C21_18 | KJ802937 | | 21700 | 22611 | pyrroloquinoline quinone |
| | | | | | biosynthesis protei |
| 182_08_C21_19 | KJ802937 | | 22697 | 23449 | pyrroloquinoline quinone |
| | | | | | biosynthesis protei |
| 182_08_C21_20 | KJ802937 | | 23543 | 23821 | pyrroloquinoline quinone |
| | | | | | biosynthesis protein Pqq |
| 182_08_C21_21 | KJ802937 | | 23793 | 24947 | pyrroloquinoline quinone |
| | | | | | biosynthesis protei |
| 182_08_C21_22 | KJ802937 | | 24944 | 26857 | prolyl oligopeptidase family |
| | | | | | protein |
| 182_08_C21_23 | KJ802937 | | 26965 | 28128 | iron-containing alcohol |
| | | | | | dehydrogenase |
| 182_08_C21_24 | KJ802937 | | 28112 | 29809 | PAS/PAC sensor hybrid |
| | | | | | histidine kinase |
| 182_08_C21_25 | KJ802937 | | 29861 | 30205 | hypothetical protein |
| | | | | | PstZobell_17449 |
| 182_08_C21_26 | KJ802937 | | 30298 | 31551 | CzcC family heavy metal |
| | | | | | RND efflux outer membrane |
| 182_08_C21_27 | KJ802937 | | 31548 | 33035 | CzcB family heavy metal |
| | | | | | RND efflux membrane fusio |
| 182_08_C21_28 | KJ802937 | | 33032 | 36154 | CzcA family heavy metal |
| | | | | | RND efflux protein |
| 182_08_C21_29 | KJ802937 | | 36268 | 37149 | Co/Zn/Cd efflux system |
| | | | | | protein |
| 182_08_C21_30 | KJ802937 | | 37158 | 37874 | hypothetical protein |
| | | | | | PstZobell_17469 |
| 182_08_C21_31 | KJ802937 | | 38081 | 38725 | DNA-binding response |
| | | | | | regulator GacA |
| 182_08_C21_32 | KJ802937 | | 38725 | 40548 | excinuclease ABC subunit C |
| 182_08_C21_33 | KJ802937 | | 40582 | 41139 | CDP-diacylglycerol--glycerol- |
| | | | | | 3-phosphate 3-p |
| 182_08_C21_34 | KJ802937 | | 41509 | 42912 | Putative integrase |
| 182_08_C21_35 | KJ802937 | | 43055 | 44821 | thiol:disulfide interchange |
| | | | | | protein precursor |
| 182_08_C21_36 | KJ802937 | | 44972 | 45430 | metal-binding protein |
| 182_08_C21_37 | KJ802937 | | 45456 | 45728 | copper-binding protein |
| 182_08_C21_38 | KJ802937 | | 45803 | 48124 | copper-translocating P-type |
| | | | | | ATPase |
| 182_08_C21_39 | KJ802937 | | 48373 | 48675 | hypothetical protein |
| | | | | | PstZobell_02371 |
| 182_08_C21_40 | KJ802937 | | 48742 | 49071 | ferredoxin |
| 182_08_C21_41 | KJ802937 | | 49226 | 50632 | sensor protein CopS |
| 182_08_C21_42 | KJ802937 | | 50629 | 50964 | transcriptional activator CopR |
| 182_08_C21_43 | KJ802937 | | 51048 | 52028 | ISPssy, transposase |
| 182_08_C21_44 | KJ802937 | | 52163 | 52501 | transcriptional activator CopR |
| 182_08_C21_45 | KJ802937 | | 53011 | 53388 | blue (type1) copper domain- |
| | | | | | containing protein |
| 182_08_C21_46 | KJ802937 | | 53574 | 55274 | copper resistance protein A/ |
| | | | | | twin-arginine translocation |
| | | | | | pathway signal |
| 182_11_B22_1 | KJ802940 | | 1 | 972 | lipoprotein |
| 182_11_B22_2 | KJ802940 | | 965 | 1732 | surface lipoprotein |
| 182_11_B22_3 | KJ802940 | | 1825 | 2667 | pirin-like protein |
| 182_11_B22_4 | KJ802940 | | 2713 | 3651 | lipid A biosynthesis lauroyl |
| | | | | | acyltransferase |
| 182_11_B22_5 | KJ802940 | | 3810 | 4535 | septum formation inhibitor |
| 182_11_B22_6 | KJ802940 | | 4631 | 5446 | septum site-determining |
| | | | | | protein MinD |
| 182_11_B22_7 | KJ802940 | | 5443 | 5700 | cell division topological |
| | | | | | specificity factor |
| 182_11_B22_8 | KJ802940 | | 5762 | 6397 | ribosomal large subunit |
| | | | | | pseudouridine synthase A |
| 182_11_B22_9 | KJ802940 | 1 | 6517 | 7806 | putative aminopeptidase 2 |
| 182_11_B22_10 | KJ802940 | 1 | 7879 | 8841 | NAD(P)(H)-dependent |
| | | | | | oxidoreductase |
| 182_11_B22_11 | KJ802940 | 1 | 9015 | 11099 | periplasmic tail-specific |
| | | | | | protease |
| 182_11_B22_12 | KJ802940 | 1 | 11271 | 11738 | TPR repeat, SEL1 subfamily |
| | | | | | protein |
| 182_11_B22_13 | KJ802940 | 1 | 11738 | 12106 | hypothetical protein |
| | | | | | PSTAB_1345 |
| 182_11_B22_14 | KJ802940 | 1 | 12103 | 12429 | Cro/Cl family transcriptional |
| | | | | | regulator |
| 182_11_B22_15 | KJ802940 | 1 | 12586 | 13062 | hypothetical protein |
| | | | | | A458_07510 |
| 182_11_B22_16 | KJ802940 | 1 | 13390 | 13704 | helix-hairpin-helix repeat- |
| | | | | | containing compet |
| 182_11_B22_17 | KJ802940 | 1 | 13825 | 15093 | flagellar hook-associated |
| | | | | | protein FlgL |
| 182_11_B22_18 | KJ802940 | 1 | 15106 | 17121 | flagellar hook-associated |
| | | | | | protein FlgK |
| 182_11_B22_19 | KJ802940 | 1 | 17125 | 18297 | flagellar rod assembly |
| | | | | | protein/muramidase Fl |
| 182_11_B22_20 | KJ802940 | 1 | 18308 | 19408 | flagellar basal body P-ring |
| | | | | | protein |
| 182_11_B22_21 | KJ802940 | 1 | 19423 | 20118 | flagellar basal body L-ring |
| | | | | | protein |
| 182_11_B22_22 | KJ802940 | 1 | 20203 | 20988 | flagellar basal body rod |
| | | | | | protein FlgG |
| 182_11_B22_23 | KJ802940 | 1 | 21024 | 21764 | flagellar basal body rod |
| | | | | | protein FlgF |
| 182_11_B22_24 | KJ802940 | 1 | 21960 | 23546 | flagellar hook protein FlgE |
| 182_11_B22_25 | KJ802940 | 1 | 23576 | 24259 | flagellar basal body rod |
| | | | | | modification protei |
| 182_11_B22_26 | KJ802940 | 1 | 24279 | 24722 | flagellar basal body rod |
| | | | | | protein FlgC |
| 182_11_B22_27 | KJ802940 | 1 | 24734 | 25198 | flagellar basal body rod |
| | | | | | protein FlgB |
| 182_11_B22_28 | KJ802940 | 1 | 25334 | 26158 | chemotaxis protein |
| | | | | | methyltransferase CheR |
| 182_11_B22_29 | KJ802940 | 1 | 26193 | 27125 | chemotaxis protein CheV |
| 182_11_B22_30 | KJ802940 | 1 | 27217 | 27957 | flagellar basal body P-ring |
| | | | | | biosynthesis pro |
| 182_11_B22_31 | KJ802940 | 1 | 28071 | 28400 | negative regulator of flagellin |
| | | | | | synthesis Fl |
| 182_11_B22_32 | KJ802940 | 1 | 28436 | 28906 | FlgN family protein |
| 182_11_B22_33 | KJ802940 | 1 | 28966 | 29712 | type IV pilus assembly PilZ |
| 182_11_B22_34 | KJ802940 | 1 | 30179 | 30391 | hypothetical protein |
| | | | | | A458_07350 |
| 182_11_B22_35 | KJ802940 | 1 | 30436 | 30621 | hypothetical protein |
| | | | | | PSTAB_1320 |
| 182_11_B22_36 | KJ802940 | 1 | 30849 | 31166 | alginate biosynthesis |
| | | | | | transcriptional activa |
| 182_11_B22_37 | KJ802940 | | 31468 | 32604 | oxaloacetate decarboxylase |
| | | | | | subunit beta |
| 182_11_B22_38 | KJ802940 | | 32615 | 34393 | pyruvate carboxylase subunit B |
| 182_11_B22_39 | KJ802940 | | 34416 | 34658 | sodium pump decarboxylase, |
| | | | | | gamma subunit |
| 182_11_B22_40 | KJ802940 | | 34799 | 36235 | magnesium transporter |
| 182_11_B22_41 | KJ802940 | | 36572 | 37054 | hypothetical protein |
| | | | | | PST_1375 |
| 182_11_B22_42 | KJ802940 | | 37591 | 37776 | carbon storage regulator |
| 182_11_B22_43 | KJ802940 | | 37957 | 39195 | aspartate kinase |
| 182_11_B22_44 | KJ802940 | | 39275 | 40012 | alanyl-tRNA synthetase |
| 182_13_F13_1 | KJ802942 | | 3 | 2480 | phage integrase family |
| | | | | | protein |
| 182_13_F13_2 | KJ802942 | 1 | 2486 | 3868 | phage integrase family |
| | | | | | protein |
| 182_13_F13_3 | KJ802942 | 1 | 4026 | 4151 | oxygen-independent |
| | | | | | coproporphyrinogen III |
| | | | | | oxidase |
| 182_13_F13_4 | KJ802942 | 1 | 4183 | 4803 | TetR family transcriptional |
| | | | | | regulator |
| 182_13_F13_5 | KJ802942 | 1 | 4879 | 6012 | class V aminotransferase |
| 182_13_F13_6 | KJ802942 | 1 | 6233 | 7627 | aromatic amino acid transport |
| | | | | | protein AroP1 |
| 182_13_F13_7 | KJ802942 | 1 | 7739 | 8524 | hydrolase, TatD family |
| 182_13_F13_8 | KJ802942 | 1 | 8628 | 8984 | type 4 fimbrial biogenesis |
| | | | | | protein PilZ |
| 182_13_F13_9 | KJ802942 | 1 | 9016 | 10002 | DNA polymerase III subunit |
| | | | | | delta′ |
| 182_13_F13_10 | KJ802942 | 1 | 9995 | 10627 | thymidylate kinase |
| 182_13_F13_11 | KJ802942 | 1 | 10624 | 11694 | hypothetical protein |
| | | | | | PST_2618 |
| 182_13_F13_12 | KJ802942 | 1 | 11691 | 12512 | 4-amino-4-deoxychorismate |
| | | | | | lyase |
| 182_13_F13_13 | KJ802942 | 1 | 12509 | 13753 | 3-oxoacyl-(acyl carrier |
| | | | | | protein) synthase II |
| 182_13_F13_14 | KJ802942 | 1 | 13926 | 14162 | acyl carrier protein |
| 182_13_F13_15 | KJ802942 | 1 | 14355 | 15098 | 3-ketoacyl-ACP reductase |
| 182_13_F13_16 | KJ802942 | 1 | 15113 | 16051 | malonyl-CoA- |
| 182_13_F13_17 | KJ802942 | 1 | 16115 | 17185 | plsX gene product |
| 182_13_F13_18 | KJ802942 | 1 | 17189 | 17371 | 50S ribosomal protein L32 |
| 182_13_F13_19 | KJ802942 | 1 | 17384 | 17911 | metal-binding protein |
| 182_13_F13_20 | KJ802942 | 1 | 18015 | 18593 | Maf-like protein |
| 182_13_F13_21 | KJ802942 | 1 | 18604 | 19587 | signal peptide peptidase |
| 182_13_F13_22 | KJ802942 | 1 | 19577 | 20263 | HAD superfamily hydrolase |
| 182_13_F13_23 | KJ802942 | 1 | 20256 | 21209 | ribosomal large subunit |
| | | | | | pseudouridine synthase |
| 182_13_F13_24 | KJ802942 | | 21768 | 24965 | ribonuclease E |
| 182_13_F13_25 | KJ802942 | | 25357 | 26376 | UDP-N- |
| | | | | | acetylenolpyruvoylglucosamine |
| | | | | | reductase |
| 182_13_F13_26 | KJ802942 | | 26373 | 26537 | protein-tyrosine-phosphatase |
| 182_16_E12_1 | KJ802943 | 1 | 2 | 382 | putative secreted protein |
| 182_16_E12_2 | KJ802943 | 1 | 476 | 1171 | hypothetical protein |
| 182_16_E12_3 | KJ802943 | 1 | 1168 | 2310 | methyl-accepting chemotaxis |
| | | | | | protein |
| 182_16_E12_4 | KJ802943 | 1 | 2468 | 3217 | AraC family transcriptional |
| | | | | | regulator |
| 182_16_E12_5 | KJ802943 | 1 | 3254 | 4891 | methyl-accepting chemotaxis |
| | | | | | sensory transduc |
| 182_16_E12_6 | KJ802943 | 1 | 5214 | 8381 | putA gene product |
| 182_16_E12_7 | KJ802943 | 1 | 8682 | 10169 | hypothetical protein |
| | | | | | BN5_00960 |
| 182_16_E12_8 | KJ802943 | 1 | 10278 | 11519 | NADH:flavin oxidoreductase |
| 182_16_E12_9 | KJ802943 | 1 | 11752 | 12867 | response regulator |
| 182_16_E12_10 | KJ802943 | 1 | 12901 | 16056 | Multidrug resistance protein |
| 182_16_E12_11 | KJ802943 | 1 | 16058 | 17143 | RND family efflux transporter |
| | | | | | MFP subunit |
| 182_16_E12_12 | KJ802943 | 1 | 17146 | 17805 | HTH-type transcriptional |
| | | | | | regulator betl |
| 182_16_E12_13 | KJ802943 | 1 | 18244 | 19002 | conserved hypothethical |
| | | | | | protein, SAM-dependent m |
| 182_16_E12_14 | KJ802943 | 1 | 19089 | 20378 | FAD-dependent |
| | | | | | oxidoreductase |
| 182_16_E12_15 | KJ802943 | 1 | 20418 | 20975 | XRE family transcriptional |
| | | | | | regulator |
| 182_16_E12_16 | KJ802943 | 1 | 21009 | 22361 | glutamine synthetase |
| 182_16_E12_17 | KJ802943 | 1 | 22412 | 22789 | MerR family transcriptional |
| | | | | | regulator |
| 182_16_E12_18 | KJ802943 | 1 | 22844 | 23422 | NADPH-dependent reductase |
| 182_16_E12_19 | KJ802943 | 1 | 23588 | 24127 | hypothetical protein |
| | | | | | PST_2845 |
| 182_16_E12_20 | KJ802943 | 1 | 24085 | 24297 | hypothetical protein |
| 182_16_E12_21 | KJ802943 | 1 | 24435 | 25718 | glycine/D-amino acid oxidase |
| 182_16_E12_22 | KJ802943 | | 26204 | 28126 | threonyl-tRNA synthetase |
| 182_16_E12_23 | KJ802943 | | 28144 | 28677 | translation initiation factor IF-3 |
| 182_16_E12_24 | KJ802943 | | 28738 | 28932 | 50S ribosomal protein L35 |
| 182_16_E12_25 | KJ802943 | | 28961 | 29317 | rplT gene product |
| 182_16_E12_26 | KJ802943 | | 29412 | 30428 | pheS gene product |
| 182_16_E12_27 | KJ802943 | | 30471 | 32273 | phenylalanyl-tRNA |
| | | | | | synthetase subunit beta |
| 182_16_J11_1 | KJ802944 | 1 | 182 | 361 | Alcohol dehydrogenase |
| | | | | | GroES domain protein |
| 182_16_J11_2 | KJ802944 | 1 | 409 | 1773 | aromatic hydrocarbon |
| | | | | | degradation outer membrane |
| | | | | | protein |
| 182_16_J11_3 | KJ802944 | 1 | 1897 | 3249 | methyl-accepting chemotaxis |
| | | | | | transducer/PAS protein |
| 182_16_J11_4 | KJ802944 | 2 | 3749 | 4765 | Glycosyl hydrolase, BNR |
| | | | | | repeat |
| 182_16_J11_5 | KJ802944 | 2 | 4776 | 7232 | RND superfamily exporter |
| 182_16_J11_6 | KJ802944 | 2 | 7277 | 9166 | cox2 cytochrome oxidase |
| | | | | | subunit |
| 182_16_J11_7 | KJ802944 | 2 | 9185 | 10489 | hypothetical protein |
| 182_16_J11_8 | KJ802944 | 2 | 10588 | 12135 | methyl-accepting chemotaxis |
| | | | | | protein, PAS domain S-box |
| 182_16_J11_9 | KJ802944 | 2 | 12240 | 13907 | malonate decarboxylase, |
| | | | | | alpha subunit |
| 182_16_J11_10 | KJ802944 | 2 | 13907 | 14782 | triphosphoribosyl-dephospho- |
| | | | | | CoA synthase |
| 182_16_J11_11 | KJ802944 | 2 | 14785 | 15084 | malonate decarboxylase |
| | | | | | subunit delta |
| 182_16_J11_12 | KJ802944 | 2 | 15077 | 15943 | mdcD gene product |
| 182_16_J11_13 | KJ802944 | 2 | 15940 | 16725 | malonate decarboxylase, |
| | | | | | gamma subunit |
| 182_16_J11_14 | KJ802944 | 2 | 16798 | 17415 | phosphoribosyl-dephospho- |
| | | | | | CoA transferase |
| 182_16_J11_15 | KJ802944 | 2 | 17412 | 18338 | malonyl CoA-acyl carrier |
| | | | | | protein transacylas |
| 182_16_J11_16 | KJ802944 | 2 | 18463 | 18885 | malonate transporter, MadL |
| | | | | | subunit |
| 182_16_J11_17 | KJ802944 | 2 | 18891 | 19655 | malonate transporter subunit |
| | | | | | MadM |
| 182_16_J11_18 | KJ802944 | 2 | 20092 | 21351 | FAD-dependent |
| | | | | | oxidoreductase |
| 182_16_J11_19 | KJ802944 | 2 | 21665 | 22582 | LysR family transcriptional |
| | | | | | regulator |
| 182_16_J11_20 | KJ802944 | 2 | 22641 | 23282 | hypothetical protein |
| | | | | | A458_00600 |
| 182_16_J11_21 | KJ802944 | 2 | 23297 | 23848 | RNA polymerase sigma factor |
| 182_16_J11_22 | KJ802944 | 2 | 24041 | 24313 | hypothetical protein |
| | | | | | A458_00610 |
| 182_16_J11_23 | KJ802944 | 2 | 24330 | 25157 | hypothetical protein |
| | | | | | PstZobell_06533 |
| 182_16_J11_24 | KJ802944 | 2 | 25154 | 25930 | hypothetical protein |
| | | | | | PstZobell_06538 |
| 182_16_J11_25 | KJ802944 | 2 | 25942 | 26433 | DoxX family protein |
| 182_16_J11_26 | KJ802944 | 2 | 26455 | 26661 | hypothetical protein |
| | | | | | A458_00630 |
| 182_16_J11_27 | KJ802944 | 2 | 26827 | 28341 | lipase, class 3 |
| 182_16_J11_28 | KJ802944 | | 28975 | 29718 | lipoprotein |
| 182_16_J11_29 | KJ802944 | | 29723 | 30568 | hypothetical protein |
| | | | | | A458_00645 |
| 182_16_J11_30 | KJ802944 | | 30565 | 32076 | Rhs element Vgr protein, type |
| | | | | | VI secretion system Vgr family |
| | | | | | protein |
| 182_17_09_1 | KJ802945 | 1 | 3 | 1040 | choline transport protein BetT |
| 182_17_09_2 | KJ802945 | 1 | 1127 | 2599 | glycine betaine aldehyde |
| | | | | | dehydrogenase |
| 182_17_09_3 | KJ802945 | 1 | 2614 | 4287 | choline dehydrogenase |
| 182_17_09_4 | KJ802945 | 1 | 4389 | 5711 | ribosomal protein S12 |
| | | | | | methylthiotransferase |
| 182_17_09_5 | KJ802945 | 1 | 5872 | 6744 | YesN family response |
| | | | | | regulator |
| 182_17_09_6 | KJ802945 | 1 | 7093 | 7284 | Flp pilus assembly protein, |
| | | | | | pilin Flp |
| 182_17_09_7 | KJ802945 | 1 | 7291 | 7812 | Flp pilus assembly protein, |
| | | | | | protease CpaA |
| 182_17_09_8 | KJ802945 | 1 | 7825 | 9147 | hypothetical protein |
| | | | | | PMI26_01591 |
| 182_17_09_9 | KJ802945 | 1 | 9160 | 9969 | Flp pilus assembly protein, |
| | | | | | RcpC family |
| 182_17_09_10 | KJ802945 | 1 | 10024 | 11538 | type II and III secretion |
| | | | | | system protein |
| 182_17_09_11 | KJ802945 | 1 | 11554 | 11823 | hypothetical protein |
| | | | | | YO5_15635 |
| 182_17_09_12 | KJ802945 | 1 | 11834 | 13168 | Flp pilus assembly protein |
| | | | | | TadG |
| 182_17_09_13 | KJ802945 | 1 | 13180 | 13650 | Flp pilus assembly protein |
| | | | | | TadG |
| 182_17_09_14 | KJ802945 | 1 | 13650 | 14153 | Flp pilus assembly protein |
| | | | | | TadG |
| 182_17_09_15 | KJ802945 | 1 | 14147 | 15376 | type II/IV secretion system |
| | | | | | ATPase TadZ |
| 182_17_09_16 | KJ802945 | 1 | 15366 | 16787 | type II/IV secretion system |
| | | | | | protein |
| 182_17_09_17 | KJ802945 | 1 | 16784 | 17770 | type II secretion system |
| | | | | | protein F |
| 182_17_09_18 | KJ802945 | 1 | 17781 | 18749 | type II secretion system |
| | | | | | protein; membrane p |
| 182_17_09_19 | KJ802945 | 1 | 18751 | 19794 | TPR repeat protein |
| 182_17_09_20 | KJ802945 | | 19807 | 20877 | O-antigen acetylase |
| 182_17_09_21 | KJ802945 | | 20900 | 21973 | glycosyl transferase family |
| | | | | | protein |
| 182_17_09_22 | KJ802945 | | 21985 | 22179 | hypothetical protein |
| | | | | | PSTAB_1644 |
| 182_17_09_23 | KJ802945 | | 22219 | 22557 | hypothetical protein |
| | | | | | PSTAB_1645 |
| 182_17_09_24 | KJ802945 | | 22605 | 23807 | glycoside hydrolase family |
| | | | | | protein |
| 182_17_09_25 | KJ802945 | | 23864 | 24910 | hypothetical protein |
| | | | | | PST_1752 |
| 182_17_09_26 | KJ802945 | | 24965 | 26071 | glycoside hydrolase family |
| | | | | | protein |
| 182_17_09_27 | KJ802945 | | 26277 | 27590 | hypothetical protein |
| | | | | | PstZobell_19633 |
| 182_17_09_28 | KJ802945 | | 27616 | 28866 | hypothetical protein |
| | | | | | PST_1755 |
| 182_17_09_29 | KJ802945 | | 28808 | 30172 | glycosyl transferase, group 1 |
| | | | | | family protein |
| 182_17_09_30 | KJ802945 | | 30180 | 30689 | transcriptional activator RfaH |
| 182_17_09_31 | KJ802945 | | 30744 | 31487 | hypothetical protein |
| 182_17_09_32 | KJ802945 | | 31510 | 33723 | tyrosine-protein kinase |
| 182_17_09_33 | KJ802945 | | 33773 | 34705 | glycosyl transferase family |
| | | | | | protein |
| 182_17_09_34 | KJ802945 | | 34674 | 35285 | polysaccharide biosynthesis |
| | | | | | protein |
| 182_35_020_1 | KJ802947 | | 1 | 165 | type 4 prepilin peptidase PilD |
| 182_35_020_2 | KJ802947 | 1 | 169 | 1389 | type II secretory pathway, |
| | | | | | component |
| 182_35_020_3 | KJ802947 | 1 | 1392 | 3095 | type IV-A pilus assembly |
| | | | | | ATPase PilB |
| 182_35_020_4 | KJ802947 | 1 | 3460 | 3645 | Tfp structural protein |
| 182_35_020_6 | KJ802947 | 1 | 4854 | 5261 | hypothetical protein |
| 182_35_020_7 | KJ802947 | 1 | 5262 | 6104 | hypothetical protein |
| 182_35_020_8 | KJ802947 | 1 | 6101 | 6997 | putative ABC transporter |
| | | | | | ATP-binding protein |
| 182_35_020_9 | KJ802947 | | 7083 | 8621 | bifunctional sulfate |
| | | | | | adenylyltransferase subunit |
| 182_35_020_10 | KJ802947 | | 8992 | 9909 | sulfate adenylyltransferase |
| | | | | | subunit 2 |
| 182_35_020_11 | KJ802947 | | 10095 | 10853 | dinuclear metal center |
| | | | | | protein, putative hydrolase- |
| | | | | | oxidas |
| 182_35_020_12 | KJ802947 | | 11014 | 12171 | 2-alkenal reductase |
| 182_35_020_13 | KJ802947 | | 12267 | 13313 | histidinol-phosphate |
| | | | | | aminotransferase |
| 182_35_020_14 | KJ802947 | | 13410 | 14720 | bifunctional histidinal |
| | | | | | dehydrogenase/histi |
| 182_35_020_15 | KJ802947 | | 14890 | 15522 | ATP |
| | | | | | phosphoribosyltransferase |
| | | | | | catalytic subu |
| 182_35_020_16 | KJ802947 | | 15758 | 17023 | UDP-N-acetylglucosamine 1- |
| | | | | | carboxyvinyltransferase |
| 182_35_020_17 | KJ802947 | | 17127 | 17366 | toluene-tolerance protein |
| 182_35_020_18 | KJ802947 | | 17466 | 17957 | hypothetical protein |
| | | | | | PST_1042 |
| 182_35_020_19 | KJ802947 | | 17971 | 18285 | toluene-tolerance protein |
| 182_35_020_20 | KJ802947 | | 18278 | 18925 | toluene-tolerance protein |
| 182_35_020_21 | KJ802947 | | 18937 | 19395 | toluene tolerance ABC |
| | | | | | transporter periplasmi |
| 182_35_020_22 | KJ802947 | | 19395 | 20192 | toluene tolerance ABC efflux |
| | | | | | transporter, pe |
| 182_35_020_23 | KJ802947 | | 20185 | 21000 | toluene tolerance ABC efflux |
| | | | | | transporter, AT |
| 182_35_020_24 | KJ802947 | | 21282 | 22256 | hypothetical protein |
| | | | | | A458_16580 |
| 182_35_020_25 | KJ802947 | | 22256 | 22780 | Yrbl family phosphatase |
| 182_35_020_26 | KJ802947 | | 22789 | 23361 | hypothetical protein |
| | | | | | A458_16590 |
| 182_35_020_27 | KJ802947 | | 23348 | 23893 | OstA family protein |
| 182_35_020_28 | KJ802947 | | 23893 | 24618 | hypothetical protein |
| 182_35_020_29 | KJ802947 | | 24764 | 26272 | sigma factor sigma-54 |
| 182_35_020_30 | KJ802947 | | 26347 | 26655 | Sigma54 modulation protein |
| 182_35_020_31 | KJ802947 | | 26663 | 27127 | phosphotransferase enzyme |
| | | | | | IIA |
| 182_35_020_32 | KJ802947 | | 27143 | 28000 | glmZ(sRNA)-inactivating |
| | | | | | NTPase |
| 182_35_020_33 | KJ802947 | | 28015 | 28287 | phosphotransferase system, |
| | | | | | phosphocarrier pr |
| 182_35_020_34 | KJ802947 | | 28340 | 29686 | PmbA protein |
| 182_35_020_35 | KJ802947 | | 29799 | 30320 | hypothetical protein |
| | | | | | A458_16635 |
| 182_35_020_36 | KJ802947 | | 30396 | 31838 | peptidase U62, modulator of |
| | | | | | DNA gyrase |
| 182_35_020_37 | KJ802947 | | 31841 | 32551 | carbon-nitrogen hydrolase |
| | | | | | family protein |
| 182_42_K21_1 | KJ802948 | 1 | 2 | 1057 | acyl-CoA dehydrogenase |
| | | | | | domain-containing protein |
| 182_42_K21_2 | KJ802948 | 1 | 1472 | 2119 | peptide methionine sulfoxide |
| | | | | | reductase |
| 182_42_K21_3 | KJ802948 | 1 | 2231 | 4903 | PAS/PAC and GAF sensor- |
| | | | | | containing |
| 182_42_K21_4 | KJ802948 | 1 | 5014 | 5541 | TPR repeat-containing protein |
| 182_42_K21_5 | KJ802948 | 1 | 5650 | 7656 | dihydrolipoamide |
| | | | | | acetyltransferase |
| 182_42_K21_6 | KJ802948 | 1 | 7681 | 10326 | pyruvate dehydrogenase |
| | | | | | subunit E1 |
| 182_42_K21_7 | KJ802948 | 1 | 10593 | 13538 | glutamate-ammonia-ligase |
| | | | | | adenylyltransferase |
| 182_42_K21_8 | KJ802948 | 1 | 13589 | 14512 | branched-chain amino acid |
| | | | | | aminotransferase |
| 182_42_K21_9 | KJ802948 | 1 | 14591 | 15625 | heptosyltransferase II |
| 182_42_K21_10 | KJ802948 | 1 | 15626 | 16627 | lipopolysaccharide |
| | | | | | heptosyltransferase I |
| 182_42_K21_11 | KJ802948 | 1 | 16627 | 17748 | waaG gene product |
| 182_42_K21_12 | KJ802948 | 1 | 17792 | 18598 | lipopolysaccharide core |
| | | | | | biosynthesis protein |
| 182_42_K21_13 | KJ802948 | 1 | 18598 | 19332 | lipopolysaccharide kinase |
| 182_42_K21_14 | KJ802948 | 1 | 19329 | 20072 | lipopolysaccharide kinase |
| 182_42_K21_15 | KJ802948 | 1 | 20072 | 21517 | serine/threonine protein |
| | | | | | kinase |
| 182_42_K21_16 | KJ802948 | 1 | 21530 | 23284 | carbamoyl transferase |
| 182_42_K21_17 | KJ802948 | 1 | 23271 | 24383 | group 1 glycosyl transferase |
| 182_42_K21_18 | KJ802948 | 1 | 24461 | 25189 | putative acetyltransferase |
| 182_42_K21_19 | KJ802948 | 1 | 25186 | 26145 | hypothetical protein |
| 182_42_K21_20 | KJ802948 | 1 | 26148 | 27020 | hypothetical protein |
| | | | | | PSTAB_3787 |
| 182_42_K21_21 | KJ802948 | 1 | 27024 | 27782 | hypothetical protein |
| | | | | | PSTAB_3786 |
| 182_42_K21_22 | KJ802948 | 1 | 27779 | 28930 | hypothetical protein |
| | | | | | PSTAB_3785 |
| 182_42_K21_23 | KJ802948 | 1 | 28955 | 29572 | Ttg8 |
| 182_42_K21_24 | KJ802948 | 1 | 29687 | 31495 | lipid A ABC exporter, fused |
| | | | | | ATPase and inner |
| 182_42_K21_25 | KJ802948 | 1 | 31495 | 32391 | Mig-14 family protein |
| 182_42_K21_26 | KJ802948 | 1 | 32395 | 33789 | LmbE family protein |
| 182_42_K21_27 | KJ802948 | 1 | 33870 | 35291 | rfaE gene product |
| 182_42_K21_28 | KJ802948 | 1 | 35326 | 36213 | hypothetical protein |
| | | | | | PST_3822 |
| 182_42_K21_29 | KJ802948 | 1 | 36299 | 37108 | putative oxidoreductase, aryl- |
| | | | | | alcohol dehydro |
| 182_42_K21_30 | KJ802948 | 1 | 37105 | 38280 | oxidoreductase, FAD-binding |
| | | | | | protein |
| 182_42_K21_31 | KJ802948 | 1 | 38273 | 38671 | multidrug efflux SMR |
| | | | | | transporter |
| 182_42_K21_32 | KJ802948 | 1 | 38766 | 39464 | 3-deoxy-D-manno- |
| | | | | | octulosonic-acid transferase |
| 183_01_D18_1 | KJ802949 | 2 | 1 | 813 | Alcohol dehydrogenase zinc- |
| | | | | | binding domain |
| 183_01_D18_2 | KJ802949 | 2 | 859 | 2646 | acyl-CoA dehydrogenase |
| 183_01_D18_3 | KJ802949 | 2 | 2673 | 3263 | nitroreductase |
| 183_01_D18_4 | KJ802949 | 2 | 3476 | 4375 | resorcinol hydroxylase small |
| | | | | | subunit |
| 183_01_D18_5 | KJ802949 | 2 | 4438 | 5319 | 6-phosphogluconate |
| | | | | | dehydrogenase NAD-binding |
| 183_01_D18_7 | KJ802949 | 2 | 5769 | 6632 | Enoyl-CoA |
| | | | | | hydratase/isomerase |
| 183_01_D18_8 | KJ802949 | 2 | 6692 | 8170 | aldehyde dehydrogenase |
| 183_01_D18_9 | KJ802949 | 2 | 8195 | 9181 | Dehydrogenase E1 |
| | | | | | component superfamily protei |
| 183_01_D18_10 | KJ802949 | 2 | 9196 | 10179 | Transketolase, C-terminal |
| | | | | | domain protein |
| 183_01_D18_11 | KJ802949 | 2 | 10189 | 11457 | 2-oxo acid dehydrogenases |
| | | | | | acyltransferase (ca |
| 183_01_D18_12 | KJ802949 | 2 | 11466 | 12878 | dihydrolipoyl dehydrogenase |
| 183_01_D18_13 | KJ802949 | 2 | 12892 | 13329 | acyl-CoA hydrolase |
| 183_01_D18_14 | KJ802949 | 2 | 13364 | 15748 | Putative bifunctional protein |
| | | | | | 3-hydroxyacyl-C |
| 183_01_D18_15 | KJ802949 | 2 | 15758 | 16954 | acetyl-CoA acetyltransferase |
| 183_01_D18_16 | KJ802949 | 2 | 16959 | 17375 | thioesterase |
| 183_01_D18_17 | KJ802949 | 2 | 17632 | 18285 | transcriptional regulator, TetR |
| | | | | | family |
| 183_01_D18_18 | KJ802949 | 2 | 18317 | 19375 | hypothetical protein |
| 183_01_D18_19 | KJ802949 | 2 | 19388 | 20749 | Protein of unknown function |
| | | | | | (DUF1329) |
| 183_01_D18_20 | KJ802949 | 2 | 20878 | 21975 | putative photosystem II |
| | | | | | stability/assembly fa |
| 183_01_D18_21 | KJ802949 | 2 | 21975 | 24422 | putative RND superfamily |
| | | | | | exporter |
| 183_01_D18_22 | KJ802949 | 2 | 24441 | 25466 | hypothetical protein |
| | | | | | AZKH_p0596 |
| 183_01_D18_23 | KJ802949 | 2 | 25543 | 26712 | major facilitator transporter |
| 183_01_D18_24 | KJ802949 | 2 | 27088 | 27576 | integrase catalytic region |
| | | | | | protein |
| 183_01_D18_25 | KJ802949 | 1 | 27956 | 28321 | putative type III effector Hop |
| | | | | | protein |
| 183_01_D18_26 | KJ802949 | 1 | 28318 | 28557 | Integrating conjugative |
| | | | | | element protein |
| 183_01_D18_27 | KJ802949 | 1 | 28580 | 28948 | integrating conjugative |
| | | | | | element |
| 183_01_D18_28 | KJ802949 | 1 | 28961 | 29371 | conjugative transfer region |
| | | | | | protein |
| 183_01_D18_29 | KJ802949 | 1 | 29451 | 30143 | integrating conjugative |
| | | | | | element protein |
| 183_01_D18_30 | KJ802949 | 1 | 30140 | 31054 | putative secreted protein |
| 183_01_D18_31 | KJ802949 | 1 | 31044 | 32477 | integrating conjugative |
| | | | | | element protein |
| 183_01_D18_32 | KJ802949 | 1 | 32458 | 32892 | Conjugative transfer region |
| | | | | | lipoprotein |
| 183_01_D18_33 | KJ802949 | 1 | 32892 | 34694 | conjugative transfer ATPase |
| 183_12_O16_1 | KJ802950 | | 2 | 928 | RND efflux transporter |
| | | | | | permease |
| 183_12_O16_2 | KJ802950 | | 943 | 2001 | RND family efflux transporter |
| | | | | | MFP subunit |
| 183_12_O16_3 | KJ802950 | | 2082 | 2468 | cobalamin (vitamin B12) |
| | | | | | biosynthesis CbiX pr |
| 183_12_O16_4 | KJ802950 | | 2548 | 3348 | UBA/THIF-type NAD/FAD- |
| | | | | | binding protein |
| 183_12_O16_5 | KJ802950 | | 3356 | 4318 | Zn-dependent hydrolase |
| 183_12_O16_6 | KJ802950 | | 4506 | 5009 | single-strand binding protein |
| 183_12_O16_7 | KJ802950 | | 5067 | 6338 | major facilitator superfamily |
| | | | | | protein |
| 183_12_O16_8 | KJ802950 | | 6432 | 9317 | excinuclease ABC subunit A |
| 183_12_O16_9 | KJ802950 | | 9278 | 10324 | UDP-glucose 4-epimerase |
| 183_12_O16_10 | KJ802950 | | 10425 | 10823 | 50S ribosomal protein L17 |
| 183_12_O16_11 | KJ802950 | | 10849 | 11829 | DNA-directed RNA |
| | | | | | polymerase subunit alpha |
| 183_12_O16_12 | KJ802950 | | 11869 | 12498 | 30S ribosomal protein S4 |
| 183_12_O16_13 | KJ802950 | | 12513 | 12902 | 30S ribosomal protein S11 |
| 183_12_O16_14 | KJ802950 | | 12915 | 13277 | rpsM gene product |
| 183_12_O16_15 | KJ802950 | | 13331 | 13444 | 50S ribosomal protein L36 |
| 183_12_O16_16 | KJ802950 | | 13470 | 13688 | translation initiation factor IF-1 |
| 183_12_O16_17 | KJ802950 | | 13693 | 15018 | preprotein translocase subunit |
| | | | | | SecY |
| 183_12_O16_18 | KJ802950 | | 15034 | 15471 | 50S ribosomal protein L15 |
| 183_12_O16_19 | KJ802950 | | 15473 | 15655 | 50S ribosomal protein L30 |
| 183_12_O16_20 | KJ802950 | | 15659 | 16183 | 30S ribosomal protein S5 |
| 183_12_O16_21 | KJ802950 | | 16196 | 16549 | 50S ribosomal protein L18 |
| 183_12_O16_22 | KJ802950 | | 16561 | 17094 | rplF gene product |
| 183_12_O16_23 | KJ802950 | | 17105 | 17500 | 30S ribosomal protein S8 |
| 183_12_O16_24 | KJ802950 | | 17514 | 17819 | 30S ribosomal protein S14 |
| 183_12_O16_25 | KJ802950 | | 17827 | 18366 | 50S ribosomal protein L5 |
| 183_12_O16_26 | KJ802950 | | 18376 | 18693 | 50S ribosomal protein L24 |
| 183_12_O16_27 | KJ802950 | | 18705 | 19073 | 50S ribosomal protein L14 |
| 183_12_O16_28 | KJ802950 | | 19227 | 19496 | 30S ribosomal protein S17 |
| 183_12_O16_29 | KJ802950 | | 19493 | 19687 | 50S ribosomal protein L29 |
| 183_12_O16_30 | KJ802950 | | 19690 | 20106 | 50S ribosomal protein L16 |
| 183_12_O16_31 | KJ802950 | | 20106 | 20876 | 30S ribosomal protein S3 |
| 183_12_O16_32 | KJ802950 | | 20886 | 21215 | 50S ribosomal protein L22 |
| 183_12_O16_33 | KJ802950 | | 21230 | 21505 | 30S ribosomal protein S19 |
| 183_12_O16_34 | KJ802950 | | 21516 | 22343 | 50S ribosomal protein L2 |
| 183_12_O16_35 | KJ802950 | | 22350 | 22655 | 50S ribosomal protein L23 |
| 183_12_O16_36 | KJ802950 | | 22652 | 23272 | 50S ribosomal protein L4 |
| 183_12_O16_37 | KJ802950 | | 23283 | 23921 | 50S ribosomal protein L3 |
| 183_12_O16_38 | KJ802950 | | 24034 | 24345 | 30S ribosomal protein S10 |
| 183_12_O16_39 | KJ802950 | | 24432 | 25622 | elongation factor Tu |
| 183_12_O16_40 | KJ802950 | | 25673 | 27772 | elongation factor G |
| 183_12_O16_41 | KJ802950 | | 27880 | 28347 | rpsG gene product |
| 183_12_O16_42 | KJ802950 | | 28381 | 28758 | 30S ribosomal protein S12 |
| 183_12_O16_43 | KJ802950 | | 28893 | 32855 | DNA-directed RNA |
| | | | | | polymerase subunit beta′ |
| 183_21_D14_1 | KJ802951 | 1 | 2 | 862 | HAD-superfamily hydrolase, |
| | | | | | subfamily IA, vari |
| 183_21_D14_2 | KJ802951 | 1 | 908 | 1774 | integral membrane protein |
| 183_21_D14_3 | KJ802951 | 1 | 1771 | 1983 | conserved hypothetical |
| | | | | | protein |
| 183_21_D14_4 | KJ802951 | 1 | 2033 | 2692 | glutathione S-transferase-like |
| | | | | | protein |
| 183_21_D14_5 | KJ802951 | 1 | 2799 | 4214 | RND efflux system outer |
| | | | | | membrane lipoprotein |
| 183_21_D14_6 | KJ802951 | 1 | 4318 | 5673 | RND family efflux transporter |
| | | | | | MFP subunit |
| 183_21_D14_7 | KJ802951 | 1 | 5670 | 6419 | ABC transporter related |
| | | | | | protein |
| 183_21_D14_8 | KJ802951 | 1 | 6419 | 7621 | ABC-type antimicrobial |
| | | | | | peptide transport syst |
| 183_21_D14_9 | KJ802951 | 1 | 7712 | 8917 | response regulator receiver |
| | | | | | modulated diguany PAS |
| 183_21_D14_10 | KJ802951 | 1 | 9126 | 9512 | heat shock protein Hsp20 |
| 183_21_D14_11 | KJ802951 | 1 | 9607 | 10953 | abc-type branched-chain |
| | | | | | amino acid transporte |
| 183_21_D14_12 | KJ802951 | 1 | 11016 | 11927 | alpha/beta hydrolase fold |
| | | | | | protein |
| 183_21_D14_13 | KJ802951 | 1 | 12278 | 13240 | transposase |
| | | | | | IS116/IS110/IS902 family |
| | | | | | protein |
| 183_21_D14_14 | KJ802951 | 1 | 13267 | 14127 | alpha/beta hydrolase family |
| | | | | | protein |
| 183_21_D14_15 | KJ802951 | 1 | 14137 | 15723 | acyltransferase, |
| | | | | | WS/DGAT/MGAT |
| 183_21_D14_16 | KJ802951 | 1 | 15903 | 16730 | PAS/PAC sensor-containing |
| | | | | | diguanylate |
| 183_21_D14_17 | KJ802951 | 1 | 16941 | 18035 | lytic murein transglycosylase B |
| 183_21_D14_18 | KJ802951 | 1 | 18032 | 20077 | transglutaminase-like |
| | | | | | enzyme, predicted cyste |
| 183_21_D14_19 | KJ802951 | 1 | 20160 | 21170 | hypothetical protein |
| | | | | | AradN_05929 |
| 183_21_D14_20 | KJ802951 | 1 | 21191 | 22111 | ATPase |
| 183_21_D14_21 | KJ802951 | 1 | 22154 | 23107 | histone deacetylase |
| | | | | | superfamily protein |
| 183_21_D14_22 | KJ802951 | 1 | 23126 | 23911 | enoyl-CoA |
| | | | | | hydratase/carnithine |
| | | | | | racemase |
| 183_21_D14_23 | KJ802951 | 1 | 23911 | 25233 | mechanosensitive ion |
| | | | | | channel protein MscS |
| 183_21_D14_24 | KJ802951 | 1 | 25488 | 26237 | electron transfer flavoprotein |
| | | | | | subunit alpha |
| 183_21_D14_25 | KJ802951 | 1 | 26390 | 27322 | electron transfer flavoprotein |
| | | | | | subunit alpha |
| 183_21_D14_26 | KJ802951 | 1 | 27499 | 29289 | acyl-CoA dehydrogenase |
| | | | | | domain protein |
| 183_21_D14_27 | KJ802951 | 1 | 29416 | 30372 | 2-nitropropane dioxygenase |
| 183_21_D14_28 | KJ802951 | 1 | 30482 | 32476 | acetate--CoA ligase |
| 183_21_D14_29 | KJ802951 | 1 | 33163 | 33468 | cytochrome c class I |
| 183_21_D14_30 | KJ802951 | 1 | 33570 | 33812 | conserved hypothetical |
| | | | | | protein |
| 183_21_D14_31 | KJ802951 | 1 | 34036 | 35892 | dihydroxy-acid dehydratase |
| 183_21_D14_32 | KJ802951 | | 36115 | 36525 | virulence-associated protein C |
| 183_21_D14_33 | KJ802951 | | 36525 | 36758 | Virulence-associated protein |
| 183_21_D14_34 | KJ802951 | | 36895 | 38076 | type III restriction protein res |
| | | | | | subunit |
| 183_24_C18_1 | KJ802952 | 1 | 2 | 685 | hypothetical protein |
| 183_24_C18_3 | KJ802952 | 1 | 836 | 1678 | hypothetical protein |
| | | | | | PMI14_02990 |
| 183_24_C18_4 | KJ802952 | 1 | 1777 | 2247 | lactoylglutathione lyase |
| 183_24_C18_5 | KJ802952 | 1 | 2432 | 2938 | hypothetical protein |
| | | | | | MEA186_14922 |
| 183_24_C18_6 | KJ802952 | 1 | 3443 | 4534 | biotin synthase |
| 183_24_C18_7 | KJ802952 | 1 | 4568 | 5758 | response regulator receiver |
| | | | | | modulated metal d |
| 183_24_C18_8 | KJ802952 | 1 | 5802 | 8843 | hypothetical protein |
| | | | | | AradN_03058 |
| 183_24_C18_9 | KJ802952 | 1 | 8862 | 9398 | Molybdopterin-binding protein |
| | | | | | KYG_10890 |
| 183_24_C18_10 | KJ802952 | 1 | 9640 | 9993 | alkylhydroperoxidase AhpD |
| 183_24_C18_11 | KJ802952 | 1 | 10024 | 10215 | putative transmembrane |
| | | | | | protein |
| 183_24_C18_12 | KJ802952 | 1 | 10313 | 11173 | metallo-beta-lactamase |
| | | | | | superfamily protein |
| 183_24_C18_13 | KJ802952 | 1 | 11279 | 11635 | ArsR family regulatory protein |
| 183_24_C18_14 | KJ802952 | 1 | 11638 | 12069 | hypothetical protein |
| | | | | | KYG_10920 |
| 183_24_C18_15 | KJ802952 | 1 | 12117 | 12557 | hypothetical protein |
| | | | | | KYG_10925 |
| 183_24_C18_16 | KJ802952 | 1 | 12559 | 12903 | hypothetical protein |
| | | | | | KYG_10930 |
| 183_24_C18_17 | KJ802952 | | 12934 | 14007 | site-specific recombinase |
| | | | | | XerD |
| 183_24_C18_18 | KJ802952 | | 14125 | 15120 | KfrA domain-containing |
| | | | | | protein DNA-binding d |
| 183_24_C18_19 | KJ802952 | | 15424 | 17808 | Diguanylate |
| | | | | | cyclase/phosphodiesterase |
| | | | | | domain |
| 183_24_C18_20 | KJ802952 | | 17987 | 18691 | short-chain |
| | | | | | dehydrogenase/reductase |
| | | | | | SDR |
| 183_24_C18_21 | KJ802952 | | 18782 | 20188 | mate efflux family protein |
| 183_24_C18_22 | KJ802952 | | 20185 | 20694 | MaoC-like protein |
| | | | | | dehydratase |
| 183_24_C18_23 | KJ802952 | | 20737 | 22014 | major facilitator transporter |
| 183_24_C18_24 | KJ802952 | | 22004 | 22504 | MarR family transcriptional |
| | | | | | regulator |
| 183_24_C18_25 | KJ802952 | | 22578 | 23129 | thioesterase superfamily |
| | | | | | protein |
| 183_24_C18_26 | KJ802952 | | 23126 | 23539 | lactoylglutathione lyase |
| 183_24_C18_27 | KJ802952 | | 23665 | 24270 | nicotinamidase-like amidase |
| 183_24_C18_28 | KJ802952 | | 24545 | 25135 | NLP/P60 protein |
| 183_24_C18_29 | KJ802952 | | 25183 | 25566 | hypothetical protein |
| | | | | | KYG_21454 |
| 183_24_C18_30 | KJ802952 | | 25776 | 26027 | putative membrane protein |
| 183_26_G23_1 | KJ802953 | | 1 | 1599 | cyanophycin synthetase |
| 183_26_G23_2 | KJ802953 | | 1596 | 2084 | CreA family protein |
| 183_26_G23_3 | KJ802953 | | 2175 | 2822 | DSBA oxidoreductase |
| 183_26_G23_4 | KJ802953 | | 3134 | 3418 | hypothetical protein |
| | | | | | KYG_20310 |
| 183_26_G23_5 | KJ802953 | | 3512 | 4894 | hypothetical protein |
| | | | | | PMI14_06112 |
| 183_26_G23_6 | KJ802953 | | 4943 | 6499 | glucose-6-phosphate |
| | | | | | isomerase |
| 183_26_G23_7 | KJ802953 | | 6649 | 7767 | 3-oxoacyl-ACP synthase |
| 183_26_G23_8 | KJ802953 | | 7847 | 8794 | transaldolase |
| 183_26_G23_9 | KJ802953 | | 8925 | 9770 | RpiR family transcriptional |
| | | | | | regulator |
| 183_26_G23_10 | KJ802953 | | 9877 | 10371 | PEBP family protein |
| 183_26_G23_11 | KJ802953 | | 10422 | 12326 | 5′-nucleotidase |
| 183_26_G23_12 | KJ802953 | | 12514 | 13518 | oligopeptide/dipeptide ABC |
| | | | | | transporter ATPase |
| 183_26_G23_13 | KJ802953 | | 13515 | 14495 | oligopeptide/dipeptide ABC |
| | | | | | transporter ATPase |
| 183_26_G23_14 | KJ802953 | | 14669 | 15580 | binding-protein-dependent |
| | | | | | transport systems i |
| 183_26_G23_15 | KJ802953 | | 15598 | 16815 | amidohydrolase |
| 183_26_G23_16 | KJ802953 | | 16817 | 17797 | binding-protein-dependent |
| | | | | | transport systems |
| 183_26_G23_17 | KJ802953 | | 17928 | 19508 | family 5 extracellular solute- |
| | | | | | binding protein |
| 183_26_G23_18 | KJ802953 | | 19811 | 20767 | porin |
| 183_26_G23_19 | KJ802953 | | 21084 | 21701 | ubiquinone biosynthesis |
| | | | | | protein COQ7 |
| 183_26_G23_20 | KJ802953 | | 21872 | 22321 | OsmC family protein |
| 183_26_G23_21 | KJ802953 | | 22634 | 24211 | threonine dehydratase |
| 183_26_G23_22 | KJ802953 | | 24510 | 25328 | cobalamin synthase |
| 183_26_G23_23 | KJ802953 | | 25325 | 25939 | phosphoglycerate mutase |
| 183_26_G23_24 | KJ802953 | | 25932 | 27578 | methyl-accepting chemotaxis |
| | | | | | sensory transduce |
| 183_26_G23_25 | KJ802953 | | 27764 | 29659 | thiamine biosynthesis protein |
| | | | | | ThiC |
| 183_26_G23_26 | KJ802953 | | 29919 | 30095 | hypothetical protein |
| | | | | | PMI12_02416 |
| 183_26_G23_27 | KJ802953 | | 30113 | 31036 | udp-3-0-acyl n- |
| | | | | | acetylglucosamine |
| | | | | | deacetylase |
| 183_26_G23_28 | KJ802953 | | 31147 | 32382 | cell division protein FtsZ |
| 183_26_G23_29 | KJ802953 | | 32543 | 33772 | cell division protein FtsA |
| 183_26_G23_30 | KJ802953 | | 33805 | 34593 | polypeptide-transport- |
| | | | | | associated domain-conta |
| 183_26_G23_31 | KJ802953 | | 34590 | 35576 | D-alanine/D-alanine ligase |
| 183_26_G23_32 | KJ802953 | | 35576 | 37006 | UDP-N-acetylmuramate--L- |
| | | | | | alanine ligase |
| 183_26_G23_33 | KJ802953 | | 37003 | 38088 | undecaprenyldiphospho- |
| | | | | | muramoylpentapeptide be |
| 183_52_O2_1 | KJ802957 | 1 | 1 | 5196 | hypothetical protein |
| | | | | | DelCs14_2697 |
| 183_52_O2_3 | KJ802957 | | 5430 | 5777 | putative signal peptide protein |
| 183_52_O2_4 | KJ802957 | | 6304 | 9102 | hsdR gene product |
| 183_52_O2_5 | KJ802957 | | 9115 | 10824 | hsdM gene product |
| 183_52_O2_6 | KJ802957 | | 10821 | 12416 | restriction modification |
| | | | | | system DNA specific |
| 183_52_O2_7 | KJ802957 | | 12413 | 14287 | hypothetical protein |
| | | | | | AZA_26080 |
| 183_52_O2_8 | KJ802957 | | 14287 | 15369 | hypothetical protein |
| 183_52_O2_9 | KJ802957 | | 15453 | 16082 | hypothetical protein ebA2393 |
| 183_52_O2_10 | KJ802957 | | 16057 | 17226 | transcriptional regulator |
| 183_52_O2_11 | KJ802957 | | 17223 | 18596 | hypothetical protein |
| | | | | | NCGM1179_3188 |
| 183_52_O2_12 | KJ802957 | | 18593 | 19159 | hypothetical protein ebA2389 |
| 183_52_O2_13 | KJ802957 | | 19305 | 19679 | ISxac2 transposase |
| 183_52_O2_14 | KJ802957 | | 19977 | 20654 | hypothetical protein |
| | | | | | PfISS101_1461 |
| 183_52_O2_16 | KJ802957 | | 20891 | 21238 | Uncharacterized protein y4hO |
| 183_52_O2_17 | KJ802957 | | 21385 | 21630 | prevent-host-death protein |
| 183_52_O2_18 | KJ802957 | | 21620 | 21904 | plasmid stabilization system |
| | | | | | protein |
| 183_52_O2_19 | KJ802957 | | 22164 | 22661 | DNA repair protein RadC |
| 183_52_O2_20 | KJ802957 | | 22636 | 23583 | hypothetical protein |
| | | | | | PAE2_4137 |
| 183_52_O2_21 | KJ802957 | | 23673 | 24677 | Phage-like protein |
| | | | | | endonuclease-like protein |
| 183_52_O2_22 | KJ802957 | | 24752 | 25720 | phage/plasmid-related protein |
| 183_52_O2_23 | KJ802957 | | 25827 | 26156 | hypothetical protein |
| | | | | | PMI22_03690 |
| 183_52_O2_24 | KJ802957 | | 26498 | 26983 | hypothetical protein |
| | | | | | Alide2_0008 |
| 183_52_O2_25 | KJ802957 | | 26995 | 27666 | hypothetical protein |
| | | | | | Despr_1026 |
| 183_52_O2_26 | KJ802957 | | 28373 | 29053 | hypothetical protein |
| | | | | | NiasoDRAFT_3049 |
| 183_52_O2_27 | KJ802957 | | 29252 | 29497 | prevent-host-death family |
| | | | | | protein |
| 183_52_O2_28 | KJ802957 | | 29642 | 29914 | hypothetical protein |
| | | | | | PseS9_11520 |
| 183_52_O2_29 | KJ802957 | | 30192 | 31598 | phage integrase |
| 183_52_O2_30 | KJ802957 | | 32163 | 33104 | electron transfer flavoprotein |
| | | | | | subunit alpha |
| 183_52_O2_31 | KJ802957 | | 33104 | 33853 | electron transfer flavoprotein, |
| | | | | | beta subunit |
| 183_52_O2_32 | KJ802957 | | 34019 | 34807 | enoyl-CoA |
| | | | | | hydratase/isomerase |
| 183_52_O2_33 | KJ802957 | | 34838 | 35590 | phbA2 gene product |
| 183_42_E18_1 | KJ802956 | | 364 | 645 | addiction module toxin, |
| | | | | | RelE/StbE family protein |
| 183_42_E18_2 | KJ802956 | | 635 | 883 | prevent-host-death family |
| | | | | | protein |
| 183_42_E18_3 | KJ802956 | | 1120 | 1518 | conserved hypothetical |
| | | | | | protein |
| 183_42_E18_4 | KJ802956 | | 1586 | 1930 | transcriptional regulator, ArsR |
| | | | | | family |
| 183_42_E18_5 | KJ802956 | | 2079 | 2288 | hypothetical protein |
| 183_42_E18_6 | KJ802956 | | 2303 | 2584 | Rhodanese domain protein |
| 183_42_E18_7 | KJ802956 | | 2608 | 3054 | OsmC family protein |
| 183_42_E18_8 | KJ802956 | | 3089 | 3280 | putative transmembrane |
| | | | | | protein |
| 183_42_E18_9 | KJ802956 | | 3381 | 4460 | biotin synthase |
| 183_42_E18_10 | KJ802956 | | 4924 | 5808 | universal stress protein |
| 183_42_E18_11 | KJ802956 | | 5829 | 6305 | lactoylglutathione lyase |
| 183_42_E18_12 | KJ802956 | | 6434 | 8482 | carbamoyl-phosphate |
| | | | | | synthase I chain ATP-bind |
| 183_42_E18_13 | KJ802956 | | 8504 | 10036 | carboxyl transferase |
| 183_42_E18_14 | KJ802956 | | 10079 | 11107 | LAO/AO transport system |
| | | | | | ATPase |
| 183_42_E18_15 | KJ802956 | | 11104 | 13272 | methylmalonyl-CoA mutase |
| 183_42_E18_16 | KJ802956 | | 13394 | 14029 | GntR family transcriptional |
| | | | | | regulator |
| 183_42_E18_17 | KJ802956 | | 14033 | 15367 | Ferric reductase domain |
| | | | | | protein transmembrane |
| 183_42_E18_18 | KJ802956 | | 15582 | 17420 | AMP-dependent synthetase |
| | | | | | and ligase |
| 183_42_E18_19 | KJ802956 | | 17539 | 18234 | Protein of unknown function |
| | | | | | (DUF3334) |
| 183_42_E18_20 | KJ802956 | | 18235 | 19356 | saccharopine dehydrogenase |
| 183_42_E18_21 | KJ802956 | | 19498 | 19950 | AsnC family transcriptional |
| | | | | | regulator |
| 183_42_E18_22 | KJ802956 | | 20036 | 21010 | Endonuclease/exonuclease/phosphatase |
| 183_42_E18_23 | KJ802956 | | 21061 | 21492 | hypothetical protein |
| | | | | | IMCC1989_1692 |
| 183_42_E18_24 | KJ802956 | | 22174 | 22764 | phospholipid/glycerol |
| | | | | | acyltransferase |
| 183_42_E18_25 | KJ802956 | | 22971 | 23486 | outer membrane |
| | | | | | protein/peptidoglycan-associat |
| 183_42_E18_26 | KJ802956 | | 23544 | 24134 | ChaC family protein |
| 183_42_E18_27 | KJ802956 | | 24156 | 24812 | pyridoxamine 5′-phosphate |
| | | | | | oxidase |
| 183_42_E18_28 | KJ802956 | | 24822 | 25088 | hypothetical protein |
| | | | | | AcdelDRAFT_1713 |
| 183_42_E18_29 | KJ802956 | | 25093 | 26049 | auxin efflux carrier |
| 183_42_E18_30 | KJ802956 | | 26152 | 27405 | chromate ion transporter |
| 183_42_E18_31 | KJ802956 | | 27490 | 29130 | GMP synthase, large subunit |
| 183_42_E18_32 | KJ802956 | | 29208 | 30683 | inosine-5′-monophosphate |
| | | | | | dehydrogenase |
| 183_42_E18_33 | KJ802956 | | 30760 | 31272 | hypothetical protein |
| | | | | | KYG_06529 |
| 183_42_E18_34 | KJ802956 | | 31295 | 31633 | hypothetical protein |
| | | | | | PMI14_04152 |
| 183_42_E18_35 | KJ802956 | | 31626 | 32066 | cyclase/dehydrase |
| 183_42_E18_36 | KJ802956 | | 32201 | 32674 | SsrA-binding protein |
| 183_42_E18_37 | KJ802956 | | 32775 | 33140 | secreted repeat protein |
| 183_42_E18_38 | KJ802956 | | 33160 | 33666 | RNA polymerase subunit |
| | | | | | sigma-24 |
| 182_10_L09_1 | KJ802939 | | 3 | 236 | LemA family protein |
| 182_10_L09_2 | KJ802939 | | 334 | 2166 | Heat shock protein HtpX |
| 182_10_L09_3 | KJ802939 | | 2373 | 2813 | hypothetical protein |
| | | | | | Tmz1t_2019 |
| 182_10_L09_4 | KJ802939 | | 3060 | 3941 | Putative alpha/beta- |
| | | | | | Hydrolase |
| 182_10_L09_5 | KJ802939 | | 4029 | 5195 | major facilitator transporter |
| 182_10_L09_6 | KJ802939 | | 5297 | 7114 | excinuclease ABC subunit C |
| 182_10_L09_7 | KJ802939 | | 7315 | 8460 | beta-hexosaminidase |
| 182_10_L09_8 | KJ802939 | | 8457 | 8837 | holo-acyl-carrier-protein |
| | | | | | synthase |
| 182_10_L09_9 | KJ802939 | | 8866 | 9621 | pyridoxine 5′-phosphate |
| | | | | | synthase |
| 182_10_L09_10 | KJ802939 | | 9621 | 10373 | DNA repair protein RecO |
| 182_10_L09_11 | KJ802939 | | 10389 | 11321 | GTP-binding protein Era |
| 182_10_L09_12 | KJ802939 | | 11318 | 11989 | ribonuclease III |
| 182_10_L09_13 | KJ802939 | | 11994 | 12350 | hypothetical protein |
| | | | | | Tmz1t_2313 |
| 182_10_L09_14 | KJ802939 | | 12423 | 13211 | lepB gene product |
| 182_10_L09_15 | KJ802939 | | 13260 | 15056 | GTP-binding protein LepA |
| 182_10_L09_16 | KJ802939 | | 15124 | 15399 | glutaredoxin |
| 182_10_L09_17 | KJ802939 | | 15396 | 16847 | protease Do |
| 182_10_L09_18 | KJ802939 | | 16844 | 17314 | positive regulator of sigma E, |
| | | | | | RseC/MucC |
| 182_10_L09_19 | KJ802939 | | 17311 | 18282 | sigma E regulatory protein, |
| | | | | | MucB/RseB |
| 182_10_L09_20 | KJ802939 | | 18279 | 18824 | anti sigma-E protein, RseA |
| 182_10_L09_21 | KJ802939 | 1 | 18834 | 19433 | algU gene product |
| 182_10_L09_22 | KJ802939 | 1 | 19622 | 21262 | L-aspartate oxidase |
| 182_10_L09_23 | KJ802939 | 1 | 21343 | 21852 | hypothetical protein |
| 182_10_L09_24 | KJ802939 | 1 | 21880 | 23115 | fabF1 gene product |
| 182_10_L09_25 | KJ802939 | 1 | 23217 | 23456 | acyl carrier protein |
| 182_10_L09_26 | KJ802939 | 1 | 23548 | 24297 | 3-ketoacyl-(acyl-carrier- |
| | | | | | protein) reductase |
| 182_10_L09_27 | KJ802939 | 1 | 24301 | 25230 | malonyl CoA-acyl carrier |
| | | | | | protein transacylas |
| 182_10_L09_28 | KJ802939 | 1 | 25267 | 26232 | 3-oxoacyl-(acyl carrier |
| | | | | | protein) synthase II |
| 182_10_L09_29 | KJ802939 | 1 | 26229 | 27245 | glycerol-3-phosphate |
| | | | | | acyltransferase PlsX |
| 182_10_L09_30 | KJ802939 | 1 | 27339 | 27518 | rpmF gene product |
| 182_10_L09_31 | KJ802939 | 1 | 27548 | 28072 | metal-binding protein |
| 182_10_L09_32 | KJ802939 | 1 | 28253 | 28828 | maf protein |
| 182_10_L09_33 | KJ802939 | 1 | 28825 | 29574 | uroporphyrin-III C/tetrapyrrole |
| | | | | | methyltransf |
| 182_10_L09_34 | KJ802939 | 1 | 29656 | 30312 | HAD-superfamily hydrolase |
| 182_07_C02_1 | KJ802936 | | 3 | 281 | hypothetical protein ebB27 |
| 182_07_C02_2 | KJ802936 | | 278 | 457 | hypothetical protein NE1441 |
| 182_07_C02_3 | KJ802936 | | 483 | 2396 | hypothetical protein ebA893 |
| 182_07_C02_4 | KJ802936 | | 2545 | 3435 | cysteine synthase B |
| 182_07_C02_5 | KJ802936 | | 3493 | 4668 | tetratricopeptide repeat |
| | | | | | protein |
| 182_07_C02_6 | KJ802936 | | 4674 | 4982 | hypothetical protein |
| | | | | | Tmz1t_3033 |
| 182_07_C02_7 | KJ802936 | | 5070 | 5357 | integration host factor subunit |
| | | | | | beta |
| 182_07_C02_8 | KJ802936 | | 5369 | 7072 | 30S ribosomal protein S1 |
| 182_07_C02_9 | KJ802936 | | 7161 | 9113 | bifunctional 3- |
| | | | | | phosphoshikimate 1- |
| | | | | | carboxyvin |
| 182_07_C02_10 | KJ802936 | | 9205 | 10092 | prephenate dehydrogenase |
| 182_07_C02_11 | KJ802936 | | 10106 | 11203 | histidinol-phosphate |
| | | | | | aminotransferase |
| 182_07_C02_12 | KJ802936 | | 11382 | 12449 | chorismate mutase |
| 182_07_C02_13 | KJ802936 | | 12520 | 13617 | phosphoserine |
| | | | | | aminotransferase |
| 182_07_C02_14 | KJ802936 | | 13617 | 16283 | DNA gyrase subunit A |
| 182_07_C02_15 | KJ802936 | | 16421 | 17023 | heat shock protein GrpE |
| 182_07_C02_16 | KJ802936 | | 17174 | 19090 | molecular chaperone DnaK |
| 182_07_C02_17 | KJ802936 | | 19187 | 20311 | chaperone protein DnaJ |
| 182_07_C02_18 | KJ802936 | | 20468 | 20959 | hypothetical protein |
| | | | | | AZL_009250 |
| 182_07_C02_19 | KJ802936 | | 21014 | 22399 | cysteinyl-tRNA synthetase |
| 182_07_C02_20 | KJ802936 | | 22638 | 23228 | cyclophilin type peptidyl-prolyl |
| | | | | | cis-trans i |
| 182_07_C02_21 | KJ802936 | | 23271 | 23765 | cyclophilin type peptidyl-prolyl |
| | | | | | cis-trans i |
| 182_07_C02_22 | KJ802936 | | 23812 | 24579 | IpxH gene product |
| 182_07_C02_23 | KJ802936 | | 24742 | 25392 | hypothetical protein |
| | | | | | Tmz1t_0120 |
| 182_07_C02_24 | KJ802936 | | 25550 | 26485 | purC gene product |
| 182_07_C02_25 | KJ802936 | | 26559 | 27821 | sugar phosphate permease |
| 182_07_C02_26 | KJ802936 | | 28000 | 28773 | hypothetical protein |
| | | | | | Tmz1t_1482 |
| 182_07_C02_27 | KJ802936 | | 28935 | 31043 | oligopeptidase A |
| 182_07_C02_28 | KJ802936 | | 31065 | 33251 | PAS/PAC sensor-containing |
| | | | | | diguanylate cycl |
| 182_07_C02_29 | KJ802936 | | 33248 | 33631 | methyl-accepting chemotaxis |
| | | | | | sensory transduc |
| 182_13_A07_1 | KJ802941 | | 268 | 2289 | methyl-accepting chemotaxis |
| | | | | | protein |
| 182_13_A07_2 | KJ802941 | | 2407 | 3087 | hypothetical protein |
| | | | | | A458_15285 |
| 182_13_A07_3 | KJ802941 | | 3100 | 3831 | hypothetical protein |
| | | | | | A458_15280 |
| 182_13_A07_4 | KJ802941 | | 3890 | 4246 | hypothetical protein |
| | | | | | A458_15275 |
| 182_13_A07_5 | KJ802941 | | 4461 | 6308 | sodium/sulfate symporter |
| | | | | | family protein |
| 182_13_A07_6 | KJ802941 | | 6537 | 7493 | alpha/beta hydrolase |
| 182_13_A07_7 | KJ802941 | | 7490 | 7966 | transcription elongation factor |
| 182_13_A07_8 | KJ802941 | | 8071 | 10011 | DNA topoisomerase III |
| 182_13_A07_9 | KJ802941 | | 10200 | 10412 | hypothetical protein |
| | | | | | A458_15250 |
| 182_13_A07_10 | KJ802941 | | 10540 | 11769 | proton-glutamate symporter |
| 182_13_A07_11 | KJ802941 | | 11964 | 12287 | hypothetical protein |
| | | | | | A458_15240 |
| 182_13_A07_12 | KJ802941 | | 12359 | 13084 | hypothetical protein |
| | | | | | PstZobell_07400 |
| 182_13_A07_13 | KJ802941 | | 13610 | 14536 | ABC transporter permease |
| 182_13_A07_14 | KJ802941 | | 14602 | 15708 | ABC transporter permease |
| 182_13_A07_15 | KJ802941 | | 15721 | 17277 | ABC transporter ATP-binding |
| | | | | | protein |
| 182_13_A07_16 | KJ802941 | | 17529 | 18449 | transcriptional regulator |
| 182_13_A07_17 | KJ802941 | | 18451 | 18906 | gamma- |
| | | | | | carboxymuconolactone |
| | | | | | decarboxylase |
| 182_13_A07_18 | KJ802941 | | 18917 | 19654 | short-chain dehydrogenase |
| 182_13_A07_19 | KJ802941 | | 19777 | 20247 | tRNA-specific adenosine |
| | | | | | deaminase |
| 182_13_A07_20 | KJ802941 | | 20284 | 21078 | ABC transporter permease |
| 182_13_A07_21 | KJ802941 | | 21065 | 21868 | ABC transporter ATP-binding |
| | | | | | protein |
| 182_13_A07_22 | KJ802941 | | 21873 | 22850 | hypothetical protein |
| 182_13_A07_23 | KJ802941 | | 22901 | 23650 | putative alkyl salicylate |
| | | | | | esterase |
| 182_13_A07_24 | KJ802941 | | 23643 | 24623 | non-heme iron-dependent |
| | | | | | enzyme |
| 182_13_A07_25 | KJ802941 | | 24916 | 27147 | PAS/PAC sensor hybrid |
| | | | | | histidine kinase |
| 182_13_A07_26 | KJ802941 | | 27386 | 29071 | PAS domain S-box |
| 182_13_A07_27 | KJ802941 | | 29055 | 30581 | circadian oscillation regulator |
| 182_13_A07_28 | KJ802941 | | 31019 | 32320 | putative ABC1 protein |
| 182_13_A07_29 | KJ802941 | | 32341 | 33042 | short chain |
| | | | | | dehydrogenase/reductase |
| | | | | | family oxidor |
| 182_13_A07_30 | KJ802941 | | 33015 | 35147 | PAS domain S-box |
| 182_13_A07_31 | KJ802941 | | 35567 | 37240 | gamma-glutamyltransferase |
| 182_13_A07_32 | KJ802941 | | 37373 | 37765 | glyoxalase/bleomycin |
| | | | | | resistance protein/diox |
| 182_13_A07_34 | KJ802941 | | 38308 | 38778 | hypothetical protein |
| | | | | | PST_2282 |
| 182_13_A07_35 | KJ802941 | | 39005 | 39409 | hypothetical protein |
| | | | | | Pext1s1_03389 |
| 182_13_A07_36 | KJ802941 | | 39862 | 40545 | LysR family transcriptional |
| | | | | | regulator |
| 183_29_M04_1 | KJ802954 | | 1 | 1551 | K+ potassium transporter |
| 183_29_M04_2 | KJ802954 | | 1697 | 2887 | benzoate transporter |
| 183_29_M04_3 | KJ802954 | | 2925 | 3881 | glutathione synthetase |
| 183_29_M04_4 | KJ802954 | 1 | 9978 | 10829 | integrase catalytic subunit |
| 183_29_M04_5 | KJ802954 | 1 | 10883 | 11209 | transposase is3/is911 family |
| | | | | | protein |
| 183_29_M04_6 | KJ802954 | 1 | 11257 | 11910 | DSBA oxidoreductase, Twin- |
| | | | | | arginine translocation pathway |
| | | | | | signal |
| 183_29_M04_7 | KJ802954 | 1 | 11965 | 12582 | sporulation domain-containing |
| | | | | | protein |
| 183_29_M04_8 | KJ802954 | 1 | 12597 | 14294 | arginyl-tRNA synthetase |
| 183_29_M04_9 | KJ802954 | 1 | 14348 | 14806 | hypothetical protein |
| | | | | | Acav_0473 |
| 183_29_M04_10 | KJ802954 | 1 | 14803 | 15735 | transcriptional regulator, LysR |
| | | | | | family |
| 183_29_M04_11 | KJ802954 | 1 | 15857 | 17803 | coenzyme A transferase |
| 183_29_M04_12 | KJ802954 | 1 | 18008 | 20209 | malate synthase G |
| 183_29_M04_13 | KJ802954 | 1 | 20320 | 20661 | putative monovalent |
| | | | | | cation/H+ antiporter subu |
| 183_29_M04_14 | KJ802954 | 1 | 20672 | 20947 | putative monovalent |
| | | | | | cation/H+ antiporter subu |
| 183_29_M04_15 | KJ802954 | 1 | 20944 | 21519 | putative K(+)/H(+) antiporter |
| | | | | | subunit E |
| 183_29_M04_16 | KJ802954 | 1 | 21516 | 23150 | putative monovalent |
| | | | | | cation/H+ antiporter subu |
| 183_29_M04_17 | KJ802954 | 1 | 23150 | 23542 | putative monovalent |
| | | | | | cation/H+ antiporter subu |
| 183_29_M04_18 | KJ802954 | 1 | 23598 | 26441 | putative monovalent |
| | | | | | cation/H+ antiporter subu |
| 183_29_M04_19 | KJ802954 | 1 | 27298 | 27486 | 4-oxalocrotonate tautomerase |
| 183_29_M04_20 | KJ802954 | 1 | 27661 | 29133 | emrB/QacA subfamily drug |
| | | | | | resistance transport |
| 183_29_M04_21 | KJ802954 | 1 | 29155 | 29922 | class-II glutamine |
| | | | | | amidotransferase |
| 183_29_M04_22 | KJ802954 | 1 | 29949 | 30329 | glyoxalase/bleomycin |
| | | | | | resistance protein/dioxy |
| 183_29_M04_23 | KJ802954 | 1 | 30741 | 31250 | hypothetical protein |
| | | | | | KYG_01427 |
| 183_29_M04_24 | KJ802954 | 1 | 32011 | 32952 | hypothetical protein |
| | | | | | Rfer_4013 |
| 183_29_M04_25 | KJ802954 | 1 | 33103 | 34191 | transposase, IS4 family |
| | | | | | protein |
| 183_29_M04_26 | KJ802954 | 1 | 34340 | 34744 | hypothetical protein |
| | | | | | Tmz1t_3596 |
| 183_29_M04_27 | KJ802954 | | 34968 | 37064 | hypothetical protein |
| 183_29_M04_28 | KJ802954 | | 37080 | 37703 | putative transposon resolvase |
| 183_29_M04_29 | KJ802954 | | 37795 | 40803 | transposase |
| 183_29_M04_30 | KJ802954 | | 40800 | 41213 | hypothetical protein |
| | | | | | Tmz1t_3596 |
| 183_38_D19_1 | KJ802955 | | 3 | 1949 | parvulin-like peptidyl-prolyl |
| | | | | | isomerase |
| 183_38_D19_2 | KJ802955 | | 2032 | 2817 | ABC-type antimicrobial |
| | | | | | peptide transport syst |
| 183_38_D19_3 | KJ802955 | | 2810 | 3808 | oligopeptide/dipeptide ABC |
| | | | | | transporter, ATP-b |
| 183_38_D19_4 | KJ802955 | | 3808 | 4701 | ABC-type antimicrobial |
| | | | | | peptide transport syst |
| 183_38_D19_5 | KJ802955 | | 4703 | 5722 | ABC-type antimicrobial |
| | | | | | peptide transport syst |
| 183_38_D19_6 | KJ802955 | | 5719 | 7329 | dipeptide transport system |
| | | | | | substrate-binding |
| 183_38_D19_7 | KJ802955 | | 7326 | 8423 | psp operon transcriptional |
| | | | | | activator PspF |
| 183_38_D19_8 | KJ802955 | | 8608 | 9276 | phage shock protein A |
| 183_38_D19_9 | KJ802955 | | 9302 | 9544 | phage shock protein B |
| 183_38_D19_10 | KJ802955 | | 9531 | 9941 | phage shock protein C |
| 183_38_D19_11 | KJ802955 | | 9987 | 11363 | hypothetical protein |
| | | | | | AGRI_06402 |
| 183_38_D19_12 | KJ802955 | | 11360 | 12379 | UPF0283 membrane protein |
| 183_38_D19_13 | KJ802955 | | 12512 | 13717 | methionine gamma-lyase |
| 183_38_D19_14 | KJ802955 | | 13894 | 14685 | phenylalanine 4- |
| | | | | | monooxygenase |
| 183_38_D19_15 | KJ802955 | | 14729 | 15070 | pterin-4-alpha-carbinolamine |
| | | | | | dehydratase |
| 183_38_D19_16 | KJ802955 | | 15219 | 16781 | transcriptional regulator of |
| | | | | | aroF, aroG, tyrA |
| 183_38_D19_17 | KJ802955 | | 16999 | 18072 | 4-hydroxyphenylpyruvate |
| | | | | | dioxygenase |
| 183_38_D19_18 | KJ802955 | | 18065 | 19204 | homogentisate 1,2- |
| | | | | | dioxygenase |
| 183_38_D19_19 | KJ802955 | | 19272 | 20294 | 2-keto-4-pentenoate |
| | | | | | hydratase/2-oxohepta-3-en |
| 183_38_D19_20 | KJ802955 | | 20380 | 21018 | maleylacetoacetate |
| | | | | | isomerase |
| 183_38_D19_21 | KJ802955 | | 21220 | 22398 | response regulator |
| 183_38_D19_23 | KJ802955 | | 22819 | 24411 | lytic murein transglycosylase |
| 183_38_D19_24 | KJ802955 | | 24543 | 25322 | hydroxyacylglutathione |
| | | | | | hydrolase |
| 183_38_D19_25 | KJ802955 | | 25385 | 26119 | SAM-dependent |
| | | | | | methyltransferase |
| 183_38_D19_26 | KJ802955 | | 26180 | 26626 | hypothetical protein |
| 183_38_D19_27 | KJ802955 | | 26610 | 27194 | acetyltransferase |
| 183_38_D19_28 | KJ802955 | | 27506 | 28039 | hypothetical protein |
| | | | | | Rhein_1400 |
| 183_38_D19_29 | KJ802955 | | 28188 | 29513 | DNA/RNA helicase, |
| | | | | | superfamily II |
| 183_38_D19_30 | KJ802955 | | 29799 | 30011 | cold shock protein |
| 183_38_D19_31 | KJ802955 | | 30225 | 31280 | nucleotidyltransferase/DNA |
| | | | | | polymerase involve |
| 183_38_D19_32 | KJ802955 | | 31559 | 32812 | glycine |
| | | | | | hydroxymethyltransferase |
| 183_38_D19_33 | KJ802955 | | 32881 | 33333 | transcriptional regulator NrdR |
| 183_38_D19_34 | KJ802955 | | 33337 | 34458 | riboflavin biosynthesis protein |
| | | | | | RibD |
| 183_38_D19_35 | KJ802955 | | 34461 | 35111 | riboflavin synthase |
| 183_38_D19_36 | KJ802955 | | 35160 | 36269 | 3,4-dihydroxy-2-butanone 4- |
| | | | | | phosphate synthase |
| 183_38_D19_37 | KJ802955 | | 36428 | 36892 | 6,7-dimethyl-8-ribityllumazine |
| | | | | | synthase |
| 183_38_D19_38 | KJ802955 | | 36902 | 37315 | transcription antitermination |
| | | | | | factor NusB |
| 183_38_D19_39 | KJ802955 | | 37344 | 38303 | thiamine-monophosphate |
| | | | | | kinase |
| 183_38_D19_40 | KJ802955 | | 38303 | 38779 | phosphatidylglycerophosphatase A |
| 183_38_D19_41 | KJ802955 | | 38807 | 40009 | diguanylate cyclase (GGDEF) |
| | | | | | domain-containing |
| 182_19_A11_2 | KJ802946 | | 141 | 1979 | diguanylate |
| | | | | | cyclase/phosphodiesterase |
| 182_19_A11_3 | KJ802946 | | 2349 | 3086 | FKBP-type peptidylprolyl |
| | | | | | isomerase |
| 182_19_A11_4 | KJ802946 | | 3739 | 4656 | recombination associated |
| | | | | | protein |
| 182_19_A11_5 | KJ802946 | 1 | 4756 | 6153 | flagellar hook protein FlgE |
| 182_19_A11_6 | KJ802946 | 1 | 6190 | 6873 | flagellar basal body rod |
| | | | | | modification protei |
| 182_19_A11_7 | KJ802946 | 1 | 6886 | 7329 | flgC gene product |
| 182_19_A11_8 | KJ802946 | 1 | 7332 | 7736 | flagellar basal body rod |
| | | | | | protein FlgB |
| 182_19_A11_9 | KJ802946 | 1 | 7960 | 9789 | glmS gene product |
| 182_19_A11_10 | KJ802946 | 1 | 9804 | 10820 | UDP-glucose 4-epimerase |
| 182_19_A11_11 | KJ802946 | 1 | 10845 | 12338 | glutamyl-tRNA synthetase |
| 182_19_A11_12 | KJ802946 | 1 | 12403 | 13317 | LysR family transcriptional |
| | | | | | regulator |
| 182_19_A11_13 | KJ802946 | 1 | 13422 | 14453 | secretion protein HlyD family |
| | | | | | protein |
| 182_19_A11_14 | KJ802946 | 1 | 14446 | 15981 | EmrB/QacA family drug |
| | | | | | resistance transporter |
| 182_19_A11_15 | KJ802946 | 1 | 16187 | 17467 | glycine/D-amino acid oxidase |
| 182_19_A11_16 | KJ802946 | 1 | 17545 | 17946 | hypothetical protein |
| | | | | | A471_09819 |
| 182_19_A11_17 | KJ802946 | 1 | 17946 | 18896 | TPR repeat-containing protein |
| 182_19_A11_18 | KJ802946 | 1 | 19113 | 20759 | nitrite reductase |
| 182_19_A11_19 | KJ802946 | 1 | 20832 | 21188 | cytochrome c551/c552 |
| 182_19_A11_20 | KJ802946 | 1 | 21268 | 21870 | tetraheme protein NirT |
| 182_19_A11_21 | KJ802946 | 1 | 21922 | 22800 | denitrification system |
| | | | | | component cytochrome |
| 182_19_A11_22 | KJ802946 | 1 | 22859 | 24058 | TPR repeat-containing protein |
| 182_19_A11_23 | KJ802946 | 1 | 24273 | 25292 | tRNA-dihydrouridine synthase A |
| 182_19_A11_24 | KJ802946 | 1 | 25344 | 26297 | transaldolase B |
| 182_19_A11_25 | KJ802946 | 1 | 26286 | 26774 | anti-sigma-factor antagonist |
| 182_19_A11_26 | KJ802946 | 1 | 26771 | 27961 | response regulator receiver |
| | | | | | protein |
| 182_19_A11_27 | KJ802946 | 1 | 28154 | 28459 | type IV pilus assembly PilZ |
| 182_19_A11_28 | KJ802946 | 1 | 28456 | 29169 | VacJ family lipoprotein |
| 182_19_A11_29 | KJ802946 | 1 | 29335 | 30486 | RND family efflux transporter |
| | | | | | MFP subunit |
| 182_19_A11_30 | KJ802946 | 1 | 30490 | 32436 | macB gene product |
| 182_19_A11_31 | KJ802946 | 1 | 32426 | 32797 | RND efflux system, outer |
| | | | | | membrane |
| 182_06_L14_1 | 182_08_C21_4 | | 2 | 220 | hypothetical protein |
| | | | | | PSJM300_10595 |
| 182_06_L14_2 | KJ802935 | | 234 | 911 | hypothetical protein |
| | | | | | PstZobell_17634 |
| 182_06_L14_3 | KJ802935 | | 1053 | 1565 | antirestriction protein family |
| | | | | | protein |
| 182_06_L14_4 | KJ802935 | | 1863 | 3059 | hypothetical protein |
| | | | | | PST_0625 |
| 182_06_L14_5 | KJ802935 | | 3059 | 3310 | XRE family transcriptional |
| | | | | | regulator |
| 182_06_L14_6 | KJ802935 | | 3685 | 3933 | hypothetical protein |
| | | | | | PSJM300_10590 |
| 182_06_L14_7 | KJ802935 | | 3934 | 4416 | hypothetical protein |
| | | | | | PSJM300_10585 |
| 182_06_L14_8 | KJ802935 | | 4510 | 5193 | ifsy-2 prophage protein |
| 182_06_L14_9 | KJ802935 | | 5280 | 8369 | error-prone DNA polymerase |
| 182_06_L14_10 | KJ802935 | | 8840 | 9529 | DNA-specific endonuclease I |
| 182_06_L14_11 | KJ802935 | 1 | 9671 | 11902 | PAS/PAC sensor hybrid |
| | | | | | histidine kinase |
| 182_06_L14_12 | KJ802935 | 1 | 12132 | 12932 | hypothetical protein |
| | | | | | CF510_08712 |
| 182_06_L14_14 | KJ802935 | 1 | 13469 | 13801 | hypothetical protein |
| 182_06_L14_16 | KJ802935 | 1 | 14375 | 15694 | hypothetical protein |
| | | | | | Aasi_0901 |
| 182_06_L14_17 | KJ802935 | 1 | 15676 | 16353 | hypothetical protein |
| | | | | | HMPREF9551_05665 |
| 182_06_L14_18 | KJ802935 | 1 | 16350 | 18029 | ABC-type transporter, |
| | | | | | ATPase and permease co |
| 182_06_L14_19 | KJ802935 | 1 | 18139 | 19035 | Zn-dependent hydrolase |
| 182_06_L14_20 | KJ802935 | 1 | 19160 | 20149 | AraC family transcriptional |
| | | | | | regulator |
| 182_06_L14_21 | KJ802935 | 1 | 20256 | 20804 | isochorismatase hydrolase |
| 182_06_L14_22 | KJ802935 | 1 | 21288 | 22250 | AraC family transcriptional |
| | | | | | regulator |
| 182_06_L14_23 | KJ802935 | 1 | 22438 | 22917 | 3-demethylubiquinone-9 3- |
| | | | | | methyltransferase |
| 182_06_L14_24 | KJ802935 | 1 | 22998 | 23756 | amino-acid ABC transporter |
| | | | | | ATP-binding prote |
| 182_06_L14_25 | KJ802935 | 1 | 23756 | 24424 | cystine ABC transporter, |
| | | | | | permease protein, p |
| 182_06_L14_26 | KJ802935 | 1 | 24421 | 25215 | cystine transporter subunit |
| 182_06_L14_27 | KJ802935 | 1 | 25320 | 26324 | D-cysteine desulfhydrase |
| 182_06_L14_28 | KJ802935 | 1 | 26423 | 27145 | transcriptional activator |
| 182_06_L14_29 | KJ802935 | 1 | 27333 | 27599 | hypothetical protein |
| | | | | | PSJM300_10525 |
| 182_06_L14_30 | KJ802935 | | 28295 | 29581 | hypothetical protein |
| | | | | | Nwat_3173 |
| 182_06_L14_31 | KJ802935 | | 29600 | 31267 | conserved hypothetical |
| | | | | | protein |
| 182_06_L14_32 | KJ802935 | | 31264 | 33141 | putative chromosome |
| | | | | | segregation ATPase |
| 182_06_L14_33 | KJ802935 | | 33151 | 33663 | hypothetical protein ec01045 |

What is claimed is:

1. A construct comprising a nucleic acid wherein the nucleic acid encodes a polypeptide that is capable of increasing lignin utilization, and wherein the nucleic acid is selected from the group consisting of nucleic acids that hybridize under stringent hybridization conditions to one of SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, and 77-96, and nucleic acids encoding a polypeptide that is at least 70% identical to a polypeptide encoded by one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, and 77-96.

2. The construct of claim 1, wherein the nucleic acid is one of SEQ ID NOS: 5, 19, 31, 41 and 43.

3. The construct of claim 1, wherein the nucleic acid is one of SEQ ID NOS: 3, 15, 27, 47, and 59.

4. The construct of claim 1, wherein the nucleic acid is a SEQ ID NO: 34.

5. The construct of claim 1, wherein the nucleic acid hybridizes under stringent hybridization conditions to one of SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, and 77-96.

6. The construct of claim 5, wherein the nucleic acid hybridizes under stringent hybridization conditions to one of SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, and 75.

7. The construct of claim 5, wherein the nucleic acid hybridizes under stringent hybridization conditions to one of SEQ ID NOS. 77-96.

8. The construct of claim 5, wherein the nucleic acid is one of SEQ ID NOS: 5, 19, 31, 41 and 43.

9. The construct of claim 5, wherein the nucleic acid is one of SEQ ID NOS: 3, 15, 27, 47, and 59.

10. The construct of claim 5, wherein the nucleic acid is a SEQ ID NO: 34.

11. The construct of claim 1, wherein the nucleic acids encode a polypeptide that is at least 80% identical to a polypeptide encoded by one of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, and 77-96.

12. The construct of claim 8, wherein the nucleic acid is one of SEQ ID NOS: 5, 19, 31, 41 and 43.

13. The construct of claim 8, wherein the nucleic acid is one of SEQ ID NOS: 3, 15, 27, 47, and 59.

14. The construct of claim 8, wherein the nucleic acid is a SEQ ID NO: 34.

15. The construct of claim 1, wherein the nucleic acids encode a polypeptide that is at least 95% identical to a polypeptide encoded by one of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, and 77-96.

16. A nucleic acid construct comprising a nucleic acid encoding a non-heme bacterial or archaeal oxidoreductase that binds Fe/Cu/Zn/Mn and utilizes a lignin or a lignin transformation product as a substrate and a nucleic acid encoding one or more bacterial proteins from functional classes (a) to (e): (a) a co-substrate generation; (b) a protein secretion; (c) a small molecule, a breakdown product, a bacterial efflux pump, or a related transmembrane protein, (d) a motility and a protein secretion; and (e) a signal transduction or a transcriptional regulation.

17. The construct of claim 7, wherein the nucleic acid encoding the oxidoreductase hybridizes under stringent hybridization conditions with a nucleic acid selected from the group consisting of SEQ ID NO: 1, 11, 13, 23, 29, 35, 37, 49, 55, 61, 63, 67, 69, and 71.

18. The construct of claim 8, wherein the nucleic acid encoding the bacterial protein from the protein secretion class hybridizes under stringent hybridization conditions with a nucleic acid selected from the group consisting of SEQ ID NO: 5, 19, 31, 41 and 43.

19. The construct of claim 8, wherein the nucleic acid encoding the bacterial protein from the class of the co-substrate generation hybridizes under stringent hybridization conditions with a nucleic acid selected from the group consisting of SEQ ID NO: 3, 15, 27, 47, and 59.

20. The construct of claim 8, wherein the nucleic acid encoding the bacterial protein from the small molecule transport class hybridizes under stringent hybridization conditions to SEQ ID NO: 34.
